(19) United States
(12) Patent Application Publication
(10) Pub. No.: US 2023/0055466 A1
(43) Pub. Date: Feb. 23, 2023

(54) A METHOD OF NUCLEIC ACID SEQUENCE ANALYSIS

(71) Applicant: INVIVOSCRIBE, INC., San Diego, CA (US)

(72) Inventor: Kasey Robert HUTT, San Diego, CA (US)

(73) Assignee: INVIVOSCRIBE, INC., San Diego, CA (US)

(21) Appl. No.: 17/788,984

(22) PCT Filed: Dec. 23, 2020
(86) PCT No.: PCT/US2020/066804

§ 371 Date: Jun. 24, 2022

Related U.S. Application Data
(60) Provisional application No. 62/953,270, filed on Dec. 24, 2019.

(51) Int. Cl. C12Q 1/6881; C12Q 1/6874; C12Q 1/6806; C12Q 1/6883; G16B 25/10

(52) U.S. Cl. CPC C12Q 1/6881; C12Q 1/6874; C12Q 1/6806; C12Q 1/6883; G16B 25/10; C12Q 2600/118

(57) ABSTRACT

The present disclosure provides methods of analysing the nucleotide read sequences of a nucleic acid sample of interest using high throughput bidirectional sequencing. The methods of the present disclosure are designed to work even where bidirectional sequencing produces forward and reverse reads that are not of a sufficient read length to be paired via the complementary hybridisation of overlapping sequences at the 3° end of the sequence reads. The disclosure further provides computer-implemented methods, computer-readable storage mediums and devices that implement a method for preparing nucleic acid sequence results for analysis from non-overlapping sequence reads for screening a nucleic acid sample of interest for the expression of one or more target nucleotide sequences.

### CROSS REFERENCE TO RELATED APPLICATION

This application claims the benefit of priority from U.S. Provisional Application No. 62/953,270, filed Dec. 24, 2019, the entire contents of which are incorporated herein by reference.

### FIELD OF THE INVENTION

The present invention relates generally to a method of analysing the nucleotide sequences of a nucleic acid sample of interest and, more particularly, to a method of analysing the nucleotide sequences of a nucleic acid sample of interest using high throughput bidirectional sequencing. The method of the present invention is based on the determination that even where bidirectional sequencing produces forward and reverse reads that are not of a sufficient read length to be paired via the complementary hybridisation of overlapping sequences at the 3′ end of the sequence reads, if the 3′ terminal ends of the sequence reads are removed and a defined portion of the 5′ end of the colocalised forward and reverse sequence reads are linked via a nucleic acid linker common to all linked reads, an accurate alignment and analysis of the sequencing results can be facilitated. The development of the method of the present invention is useful in a range of applications including, but not limited to, diagnosing a condition characterised by the presence of a clonal population of cells (such as a neoplastic condition) or microorganism, monitoring the progression of such a condition, predicting the likelihood of a subject's relapse from a remissive state to a disease state, assessing the effectiveness of existing therapeutic drugs and/or new therapeutic agents or immune surveillance.

### INCORPORATION BY REFERENCE OF SEQUENCE LISTING

The Sequence Listing in the ASCII text file, named as 38093WO.P41235PCUS.SeqListing.txt of 3 KB, created on Dec. 16, 2020, and submitted to the United States Patent and Trademark Office via EFS-Web, is incorporated herein by reference.

### BACKGROUND OF THE INVENTION

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification.

Bibliographic details of the publications referred to by author in this specification are collected alphabetically at the end of the description.

A clone is generally understood as a population of cells which has descended from a common precursor cell. Diagnosis and/or detection of the existence of a clonal population of cells or organisms in a subject has generally constituted a relatively problematic procedure. Specifically, a clonal population may constitute only a minor component within a larger population of cells or organisms. For example, in terms of the mammalian organism, one of the more common situations in which the detection of a clonal population of cells is required occurs in terms of the diagnosis and/or detection of neoplasms, such as cancer. However, detection of one or more clonal populations may also be important in the diagnosis of conditions such as myelodysplasia or polycythaemia vera and also in the detection of antigen driven clones generated by the immune system in the context of infection, autoimmune disease, allergy or transplantation.

If the members of the clone are characterized by a molecular marker, such as an altered sequence of DNA, then the problem of detection may be able to be translated into the problem of detecting a population of molecules which all have the same molecular sequence within a larger population of molecules which have a different sequence. The level of detection of the marker molecules that can be achieved is very dependent upon the sensitivity and specificity of the detection method, but nearly always, when the proportion of target molecules within the larger population of molecules becomes small, the signal noise from the larger population makes it difficult to detect the signal from the target molecules.

A specific class of molecular markers which, although highly specific, present unique complexities in terms of its detection are those which result from genetic recombination events. Recombination of the genetic material in somatic cells involves the bringing together of two or more regions of the genome which are initially separate. It may occur as a random process but it also occurs as part of the developmental process in normal lymphoid cells.

In relation to cancer, recombination may be simple or complex. A simple recombination may be regarded as one in which two unrelated genes or regions are brought into apposition. A complex recombination may be regarded as one in which more than two genes or gene segments are recombined. The classical example of a complex recombination is the rearrangement of the immunoglobulin and T-cell receptor variable genes which occurs during normal development of lymphoid cells and which involves recombination of the V, D and J gene segments. The loci for these gene segments are widely separated in the germline but recombination during lymphoid development results in apposition of V, D and J gene segments, or V and J gene segments, with the junctions between these gene segments being characterised by small regions of insertion and deletion of nucleotides (N₁ and N₂ regions). This process occurs randomly so that each normal lymphocyte comes to bear a unique V(D)J rearrangement which may be a complete VDJ rearrangement or a VJ or DJ rearrangement, depending both on the gene which is rearranged and on the nature of the rearrangement. Since a lymphoid cancer, such as acute lymphoblastic leukaemia, chronic lymphocytic leukaemia, lymphoma or myeloma, occurs as the result of neoplastic change in a single normal cell, all of the cancer cells will, at least originally, bear the junctional V(D)J rearrangement originally present in the founder cell. Subclones may arise during expansion of the neoplastic population and further V(D)J rearrangements may occur in them.

The unique DNA sequences resulting from recombination and which are present in a cancer clone or subclone provide a unique genetic marker which can be used to monitor the response to treatment and to make decisions on therapy. Monitoring of the clone can be performed by a range of techniques including PCR, flow cytometry or next-generation sequencing, each of which present a range of strengths and weaknesses.

Although PCR revolutionized the analysis of DNA by virtue of the ability to exponentially amplify a target DNA, in particular DNA present in low starting copy number, traditional sequencing methods, such as Sanger sequencing, were still slow. This made the large scale sequence based analysis of PCR amplified patient DNA virtually impossible. The advent of next generation sequencing revolutionised sequencing based analysis by providing a high throughput approach to DNA sequencing. This meant that the turnaround time and cost associated with traditional sequencing was reduced and nucleic acid sequencing became available on a large scale. When coupled with the evolution of PCR to solid phase bridge amplification based colony generation, the significantly more sophisticated, informative and much more accurate information provided by nucleic acid sequencing analysis became routinely available.

There are a wide range of both DNA library amplification methods and next generation sequencing methods which have been developed. For example, three of the more common PCR-based amplification methods are emulsion PCR, rolling circle amplification and solid-phase amplification.

In emulsion PCR methods, a DNA library is initially generated. Single-stranded DNA fragments are attached to the surface of beads with adaptors or linkers, and one bead is attached to a single DNA fragment from the DNA library. The surface of the beads contains oligonucleotide probes with sequences that are complementary to the adaptors binding the DNA fragments. The beads are then compartmentalized into water-oil emulsion droplets. In the aqueous water-oil emulsion, each of the droplets capturing one bead is a PCR microreactor that produces amplified copies of the single DNA template.

Gridded Rolling Circle Nanoballs describes the amplification of a population of single DNA molecules by rolling circle amplification in solution followed by capture on a grid of spots sized to be smaller than the DNAs to be immobilized.

DNA colony generation (Bridge amplification) uses forward and reverse primers which are covalently attached at high-density to the slide of a flow cell. The ratio of the primers to the template on the support defines the surface density of the amplified clusters. The flow cell is exposed to reagents for polymerase-based extension, and priming occurs as the free/distal end of a ligated fragment “bridges” to a complementary oligonucleotide on the surface. Repeated denaturation and extension results in localized amplification of DNA fragments in millions of separate locations across the flow cell surface. Solid-phase amplification produces 100-200 million spatially separated template clusters, providing free ends to which a universal sequencing primer is then hybridized to initiate the sequencing reaction.

In terms of next generation sequencing approaches, four well known technologies include pyrosequencing, sequencing by reversible terminator chemistry, sequencing-by-ligation mediated by ligase enzymes and phospholinked fluorescent nucleotides sequencing.

Pyrosequencing is a non-electrophoretic, bioluminescence method that measures the release of inorganic pyrophosphate by proportionally converting it into visible light using a series of enzymatic reactions. Unlike other sequencing approaches that use modified nucleotides to terminate DNA synthesis, the pyrosequencing method manipulates DNA polymerase by the single addition of a dNTP in limiting amounts. Upon incorporation of the complementary dNTP, DNA polymerase extends the primer and pauses. DNA synthesis is reinitiated following the addition of the next complementary dNTP in the dispensing cycle. The order and intensity of the light peaks are recorded as flowgrams, which reveal the underlying DNA sequence.

Sequencing by reversible terminator chemistry uses reversible terminator-bound dNTPs in a cyclic method that comprises nucleotide incorporation, fluorescence imaging and cleavage. A fluorescently-labelled terminator is imaged as each dNTP is added and then cleaved to allow incorporation of the next base. These nucleotides are chemically blocked such that each incorporation is a unique event. An imaging step follows each base incorporation step, then the blocked group is chemically removed to prepare each strand for the next incorporation by DNA polymerase. This series of steps continues for a specific number of cycles, as determined by user-defined instrument settings. The 3′ blocking groups were originally conceived as either enzymatic or chemical reversal. This method has been the basis for the Solexa and Illumina machines. Sequencing by reversible terminator chemistry can be performed as a four-colour cycle such as used by Illumina/Solexa, or a one-colour cycle such as used by Helicos BioSciences. Helicos BioSciences uses “virtual terminators”, which are unblocked terminators with a second nucleoside analogue that acts as an inhibitor. These terminators incorporate the appropriate modifications for terminating or inhibiting groups so that DNA synthesis is terminated after a single base addition. Reversible terminator sequencing can be designed as either bidirectional (paired-end) sequencing or single read sequencing.

Sequencing-by-ligation mediated by ligase enzymes uses a sequence extension reaction which is not carried out by polymerases but rather by DNA ligase and either one-base-encoded probes or two-base-encoded probes. In its simplest form, a fluorescently labelled probe hybridizes to its complementary sequence adjacent to the primed template. DNA ligase is then added to join the dye-labelled probe to the primer. Non-ligated probes are washed away, followed by fluorescence imaging to determine the identity of the ligated probe. The cycle can be repeated either by using cleavable probes to remove the fluorescent dye and regenerate a 5′-PO4 group for subsequent ligation cycles (chained ligation) or by removing and hybridizing a new primer to the template (unchained ligation).

Phospholinked Fluorescent Nucleotides sequencing is a method of real-time sequencing which involves imaging the continuous incorporation of dye-labelled nucleotides during DNA synthesis. Single DNA polymerase molecules are attached to the bottom surface of individual zero-mode waveguide detectors that can obtain sequence information while phospolinked nucleotides are being incorporated into the growing primer strand. Pacific Biosciences, for example, uses a unique DNA polymerase which better incorporates phospholinked nucleotides and enables the resequencing of closed circular templates.

These technologies are available in various commercial platforms such as those summarised in Table 1, below.

| | | | | Run |
|---|---|---|---|---|
| | Template | | Max Read | Times |
| Platform | Preparation | Chemistry | length (bases) | (days) |
| Roche 454 | Clonal-emPCR | Pyrosequencing | 400 | 0.42 |
| GS FLX Titanium | Clonal-emPCR | Pyrosequencing | 400 | 0.42 |
| Illumina MiSeq | Clonal Bridge | Reversible Dye | 2 × 300 | 0.17-2.7 |
| | Amplification | Terminator | | |
| Illumina HiSeq | Clonal Bridge | Reversible Dye | 2 × 150 | 0.3-11 |
| | Amplification | Terminator | | |
| Ilumina Genome | Clonal Bridge | Reversible Dye | 2 × 150 | 2-14 |
| Analyzer IIX | Amplification | Terminator | | |
| Life Technologies | Clonal-emPCR | Oligonucleotide | 20-45 | 4-7 |
| SOLiD4 | | 8-mer Chained | | |
| | | Ligation | | |
| Life Technologies | Clonal-emPCR | Native dNTPs, | 200 | 0.5 |
| Ion Proton^{[16]} | | proton | | |
| | | detection | | |
| Complete Genomics | Gridded DNA- | Oligonucleotide | 7 × 10 | 11 |
| | nanoballs | 9-mer Unchained | | |
| | | Ligation | | |
| Helicos Biosciences | Single | Reversible Dye | 35 | 8 |
| Heliscope | Molecule | Terminator | | |
| Pacific Biosciences | Single | Phospholinked | 10,000 (N50); | 0.08 |
| SMRT | Molecule | Fluorescent | 30,000+ (max) | |
| | | Nucleotides | | |

The combination of solid phase bridge amplification of target DNA followed by reversible dye terminator bidirectional sequencing has proved to be a particularly efficient means of achieving high throughput amplification and sequencing. However, one of the limitations of bidirectional sequencing utility has been the maximum number of cycles which can be performed and which thereby limits the maximum sequence read length which can be generated. For example, the Illumina HiSeq instrument can generate 2×250 base bidirectional reads while the MiSeq instrument can generated 2×300 base bidirectional reads. The NextSeq and NovaSeq instruments both generate 2×150 base bidirectional reads. In the context of long DNA targets, such as chromosomes or other long sections of genome, the generation of what are relatively short reads are nevertheless useful since these reads can be paired (also referred to as “taped” or “stitched”) based on the complementarity of overlapping sequences at their 3′ ends, thereby generating a double stranded DNA sequence section. Each of these taped sequences can then be further aligned based on sequence overlap with other taped reads to assemble a longer stretch of genomic sequence. This alignment is often performed relative to a reference sequence. In this regard, where sequence reads do not overlap, the use of a reference sequence against which to align these reads can provide a means of analysing the reads relative to the reference sequence. However, in the absence of a sequence read relative to which an analysis can be performed, non-overlapping reads are currently of little utility other than in the context of whatever information they can provide as individual stand alone sequencing results.

In the context of some DNA target regions of interest, such as rearranged immunoglobulin (herein referred to as “Ig”) or T cell receptor (herein referred to as “TCR”) molecules, where each individual amplicon is analysed to determine whether it represents one member of a population of clonal sequences within a biological sample of interest or, alternatively, represents a residual or recurrent clonal sequence, it is usually necessary for the bidirectional sequence reads to provide sufficient forward and reverse read length such that the 3′ ends of the reads overlap and can be taped based on their complementarity, thereby providing the entire target sequence region, such as the rearranged VJ gene segments of a T or B cell, or a span of genomic DNA which potentially encompasses a mutation, chromosomal translocation site, DNA breakpoint or an inversion or indel site. Where the DNA region which is required to be amplified in order to detect this nucleotide characteristic is longer than what the chemistry of the selected instrument will enable the sequencing of, the bidirectional forward and reverse reads which are generated from the 5′ and 3′ terminal ends of such a template are unlikely to be of sufficient length to overlap and therefore cannot be taped together. Accordingly, currently available high throughput instrumentation and methodology limits the type and scope of sequencing analyses which can be performed in the context of screening for specific sequences or surveying the diversity of a DNA population of interest.

In work leading up to the present invention, it has been unexpectedly determined that even where bidirectional sequencing chemistry is insufficient to generate overlapping forward and reverse reads, it is nevertheless possible to screen a DNA sample of interest for the expression of one or more target nucleotide sequences by generating a template DNA library from the starting biological sample wherein irrespective of the length of each individual template DNA molecule, the templates have been designed such that the target nucleotide sequences are localised to the 5′ and 3′ ends of the template DNA, specifically within a 5′ or 3′ terminal nucleotide stretch which corresponds to approximately 80% of the length of the bidirectional sequence read length which has been selected for use. Accordingly, the bidirectional sequencing step will effectively sequence the target nucleotide sequence since it is localised to the region known to fall within the read length. Although these sequence reads will not comprise a read length sufficient for the forward and reverse reads to overlap, the spatial colocalization of the reads, if they have been generated from amplicons which were themselves generated on a solid phase via cluster amplification of individual template DNA molecules, provides a means to identify the likely bidirectional sequence read pairs.

However, due to the increasing likelihood of sequencing errors as a bidirectional sequencing read progresses in the 3′ direction, these reads cannot be reliably aligned and analysed using currently available analytical tools since these tools rely on the hybridisation of the overlapping 3′ ends of the paired reads to assist in distinguishing between random sequencing errors versus the presence of a SNP or point mutation. Still further, it has been unexpectedly determined that due to the fact that variability in the final sequence length between reads will occur (not all amplicons will necessarily be sequenced to the maximum theoretical read length for the selected instrument), even if the actual sequences of these reads are otherwise identical across the sequence length which is produced, these reads will nevertheless be routinely misclassified as separate and distinct sequences due simply to the differing read length. Accordingly, the combination of sequencing errors which naturally occur at the 3′ end of the sequence read, together with misclassification of reads which are of different length but otherwise identical, will result in substantial skewing of the test results.

Where traditional overlapping bidirectional sequencing reads are generated, both of the above described problems are alleviated. The issue of variation in sequence length is rendered moot since the forward and reverse reads overlap and can be hybridised based on the complementarity of the overlapping sequence, thereby generating a double stranded molecule, and the 3′ sequencing errors are easily identified and discarded (rather than being classified as a unique sequence) by virtue of the complementary paired end read which expresses the correct complementary nucleotide. Accordingly, in the absence of the generation of overlapping sequence reads, the analysis of non-overlapping reads in their original form has been determined to produce substantially erroneous results, which in the clinical setting can prove extremely problematic.

In terms of the present invention, it has been surprisingly determined that if in addition to the specific template design described herein, forward and reverse sequence reads are cleaved to remove the 3′ sequence read up to a point that the remaining read is not less than about 80% of the maximum bidirectional sequence read length which is selected for use, and the cleaved and colocalised forward and reverse bidirectional reads are linked with the sequences complementary to said reverse and forward reads, respectfully, to form a linear molecule via a linear linker sequence which is common to all the paired colocalised reads, the resulting “taped” sequence read, when aligned with other reads and/or otherwise analysed will produce a highly accurate result in relation to the presence, nature and/or diversity of the target nucleotide sequence in the DNA sample of interest. It has also been determined that in the context of immunoglobulin and TCR gene rearrangement, even where the 5′ and 3′ reads derived from two or more clusters are identical, there nevertheless remains a possibility that these reads were generated from two different template molecules where although the target sequences were the same as between these molecules, the intervening (non-amplified) sequence was different. In this situation, these reads would be classified as deriving from a common clone. However, it has now been found that in the context of rearranged VDJ gene segments, the incidence of this sequencing anomaly does not, in fact, adversely impact the sensitivity or specificity of the test results. By designing and generating the template DNA library to ensure that the target sequences are localised to the 5′ and 3′ ends of the template molecules, it is now possible to conduct high throughput next generation sequencing without necessarily having to ensure that the template DNA library fragments are of a size across which the selected bidirectional sequencing instrumentation can sequence the full length. This development has therefore now substantially widened the application of current next generation bidirectional sequencing chemistry and instrumentation such that the selection of suitable instrumentation is no longer necessarily limited by the maximum read length of a given instrument relative to the length of the DNA template of interest. Provided that the target sequences can be expressed within the 5′ and 3′ terminal DNA regions hereinbefore described, the overall length of the DNA template from which the amplicon cluster will be generated and sequenced becomes irrelevant and is no longer a limitation. Still further, the present method has also enabled the pairing and analysis of non-overlapping sequence reads without the need to perform this step relative to a reference sequence against which the individual reads are aligned.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The present invention is not to be limited in scope by the specific embodiments described herein, which are intended for the purposes of exemplification only. Functionally-equivalent products, compositions and methods are clearly within the scope of the invention, as described herein.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

The subject specification contains nucleotide sequence information prepared using the programme PatentIn Version 3.1, presented herein after the bibliography. Each nucleotide sequence is identified in the sequence listing by the numeric indicator <210> followed by the sequence identifier (e.g. <210>1, <210>2, etc). The length, type of sequence (DNA, etc) and source organism for each nucleotide sequence are indicated by information provided in the numeric indicator fields <211>, <212> and <213>, respectively. Nucleotide sequences referred to in the specification are identified by the indicator SEQ ID NO: followed by the sequence identifier (e.g. SEQ ID NO:1, SEQ ID NO:2, etc.). The sequence identifier referred to in the specification correlates to the information provided in numeric indicator field <400> in the sequence listing, which is followed by the sequence identifier (e.g. <400>1, <400>2, etc.). That is SEQ ID NO:1 as detailed in the specification correlates to the sequence indicated as <400>1 in the sequence listing.

One aspect of the present invention is directed to a method of screening a nucleic acid sample of interest for the expression of one or more target nucleotide sequences, said method comprising:

(i) spatially isolating on a solid support a library of individual template DNA molecules derived from said nucleic acid sample, which template DNA molecules have been generated such that the target nucleotide sequences are localised to the region of contiguous nucleotides at the 5′ and/or 3′ terminal ends of said template;

(ii) amplifying said spatially isolated template DNA molecules to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read; and wherein:
(1) said portion is not less than 75% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all the nucleic acid sequence results of (a) and the linker sequence is the same for all the nucleic acid sequence results of (b); and

(v) analysing the sequence result.

In another aspect there is provided a method of screening a DNA sample of interest for the expression of one or more target DNA sequences, said method comprising:

(i) spatially isolating on a solid support a library of individual template DNA molecules derived from said DNA sample, which template DNA molecules have been generated such that the target DNA sequences are localised to the region of contiguous nucleotides at the 5′ and/or 3′ terminal ends of said template;

(ii) amplifying said spatially isolated template DNA molecules to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read; and wherein:
(1) said portion is not less than 75% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all the nucleic acid sequence results of (a) and the linker sequence is the same for all the nucleic acid sequence results of (b); and

(v) analysing the sequence result.

In yet another aspect there is provided a method of screening a DNA sample comprising B and/or T cell DNA for the expression of one or more rearranged V, D or J gene segments, said method comprising:

(i) spatially isolating on a solid support a library of individual template DNA molecules derived from said DNA sample, which template DNA molecules have been generated such that said rearranged V, D or J gene segments are localised to the region of contiguous nucleotides at the 5′ and/or 3′ terminal ends of said template;

(ii) amplifying said spatially isolated template DNA molecules to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read; and wherein:
(1) said portion is not less than 75% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all nucleic acid sequence results; and

(v) analysing the sequence result.

In another embodiment, said contiguous nucleotide region of step (i) corresponds to about 80% of the maximum forward and reverse read length deliverable by the bidirectional sequencing technology selected for use in step (iii)

In another embodiment and in the context of V(D)J rearrangement, said target nucleotide sequences are the DJ or VDJ rearrangements of IgH, TCR β or TCR δ. In another embodiment said target nucleotide sequences are the VJ rearrangement of Igκ, Igλ, TCRα or TCRγ. In another embodiment said rearrangement is a kappa deleting element rearrangement.

In yet another embodiment, said target nucleotide sequences are a V gene segment region, such as a region predisposed to undergoing hypermutation and/or a J gene segment region encoding a portion of the CDR3.

In still yet another embodiment, said target nucleotide sequences are the gene segment regions encoding all or some of the V leader sequence, the V region predisposed to somatic hypermutation, IgH FR1, IgH FR2 or IgH FR3.

In yet still another embodiment, said target nucleotide sequence is the BCL1/JH translocation or BCL2/JH t(14:18).

In a further aspect there is provided a method of screening a DNA sample of interest for the expression of one or more target DNA sequences, said method comprising:

(i) spatially isolating on a glass surface a library of individual template DNA molecules derived from said DNA sample, which template DNA molecules have been generated such that the target DNA sequences are localised to the region of contiguous nucleotides at the 5′ and/or 3′ terminal ends of said template and wherein said contiguous nucleotide region corresponds to about 80% of the maximum forward and reverse read length deliverable by the bidirectional sequencing technology selected for use in step (iii);

(ii) amplifying said spatially isolated template DNA molecules to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read;
and wherein:
(1) said portion is not less than 75% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all the nucleic acid sequence results of (a) and the linker sequence is the same for all the nucleic acid sequence results of (b); and

(v) analysing the sequence result.

Preferably said glass surface is a glass slide or a flow cell.

In yet still another aspect there is provided a method of screening a DNA sample of interest for the expression of one or more target DNA sequences, said method comprising:

(i) spatially isolating on a glass surface a library of individual template DNA molecules derived from said DNA sample, which template DNA molecules have been generated such that the target DNA sequences are localised to the region of contiguous nucleotides at the 5′ and/or 3′ terminal ends of said template, wherein said contiguous nucleotide region corresponds to about 80% of the maximum forward and reverse read length deliverable by the bidirectional sequencing technology selected for use in step (iii) and wherein the terminal end of said contiguous nucleotide region expresses one or more nucleic acid sequences corresponding to indexes, barcodes, unique molecular identifiers, sequencing primer hybridisation sites and index sequencing primer hybridisation sites;

(ii) amplifying said spatially isolated template DNA molecules to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read;
and wherein:
(1) said portion is not less than 75% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all the nucleic acid sequence results of (a) and the linker sequence is the same for all the nucleic acid sequence results of (b); and

(v) analysing the sequence result.

In another further aspect there is provided a method of screening a DNA sample of interest for the expression of one or more target DNA sequences, said method comprising:

(i) spatially isolating on a glass surface a library of individual template DNA molecules derived from said DNA sample, which template DNA molecules have been generated such that the target DNA sequences are localised to the region of contiguous nucleotides at the 5′ and/or 3′ terminal ends of said template, wherein said contiguous nucleotide region corresponds to 75%, 76%, 77%, 78%, 79%, 80%, 81%, 82% or 83% of the maximum forward and reverse read length deliverable by the bidirectional sequencing technology selected for use in step (iii) and wherein the terminal end of said contiguous nucleotide region expresses one or more nucleic acid sequences corresponding to adaptors indexes, barcodes, unique molecular identifiers, sequencing primer hybridisation sites and index sequencing primer hybridisation sites;

(ii) amplifying said spatially isolated template DNA molecules to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read;
and wherein:
(1) said portion is not less than 75%, 76%, 77%, 78%, 79%, 80%, 81%, 82% or 83% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all the nucleic acid sequence results of (a) and the linker sequence is the same for all the nucleic acid sequence results of (b); and

(v) analysing the sequence result.

In one embodiment, said target DNA sequences are localised to the 120 contiguous nucleotides at the 5′ and/or 3′ terminal ends of said template but wherein up to the 20 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites.

In another embodiment, said target DNA sequences are localised to the 125 contiguous nucleotides at the 5′ and/or 3′ terminal ends of said template but wherein up to the 30 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites.

In a further aspect, there is provided a method of screening a DNA sample of interest for the expression of one or more target DNA sequences, said method comprising:

(i) spatially isolating on a glass surface a library of individual template DNA molecules derived from said DNA sample, which template DNA molecules have been generated such that the target DNA sequences are localised to the region of contiguous nucleotides at the 5′ and/or 3′ terminal ends of said template, wherein the terminal end of said contiguous nucleotide region expresses one or more nucleic acid sequences corresponding to indexes, barcodes, unique molecular identifiers, sequencing primer hybridisation sites and index sequencing primer hybridisation sites;

(ii) amplifying said spatially isolated template DNA molecules by bridge amplification to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read;
and wherein:
(1) said portion is not less than 75% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all the nucleic acid sequence results of (a) and the linker sequence is the same for all the nucleic acid sequence results of (b); and

(v) analysing the sequence result.

In yet still another aspect, there is provided a method of screening a DNA sample of interest for the expression of one or more target DNA sequences, said method comprising:

(i) spatially isolating on a glass surface a library of individual template DNA molecules derived from said DNA sample, which template DNA molecules have been generated such that the target DNA sequences are localised to the region of contiguous nucleotides at the 5′ and/or 3′ terminal ends of said template, wherein the terminal end of said contiguous nucleotide region expresses one or more nucleic acid sequences corresponding to indexes, barcodes, unique molecular identifiers, sequencing primer hybridisation sites and index sequencing primer hybridisation sites;

(ii) amplifying said spatially isolated template DNA molecules by bridge amplification to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon and wherein said bidirectional sequencing is sequencing by synthesis using reversibly terminated labelled nucleotides;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read;
and wherein:
(1) said portion is not less than 75% of the maximum forward and reverse read length deliverable by the selected bidirectional sequencing technology, (b) said portion of the reverse read contiguous sequence is the same for all reverse reads which are analysed, (c) said portion of the forward read contiguous sequence is the same for all forward reads which are analysed but may be the same or different to the reverse read portion and (d) the linker sequence is the same for all the nucleic acid sequence results of (a) and the linker sequence is the same for all the nucleic acid sequence results of (b); and

(v) analysing the sequence result.

In accordance with the above aspects, in one embodiment said glass surface is a glass slide or a flow cell.

In still another embodiment said contiguous nucleotide region of step (i) corresponds to about 80% of the maximum forward and reverse read length deliverable by the bidirectional sequencing technology selected for use in step (iii)

In another embodiment said nucleic sample of interest comprises B and/or T cell DNA and said one or more target nucleotide sequences are one or more rearranged V, D or J gene segments.

In yet another embodiment said target nucleotide sequences are the DJ or VDJ rearrangements of IgH, TCR β or TCR δ or the VJ rearrangement of Igκ, Igλ, TCRα or TCRγ. In still another embodiment, said rearrangement is a kappa deleting element rearrangement.

In still yet another embodiment, said target nucleotide sequences are a V gene segment region, such as a region predisposed to undergoing hypermutation and/or a J gene segment region encoding a portion of the CDR3.

In yet still another embodiment, said target nucleotide sequences are the gene segment regions encoding all or some of the V leader sequence, the V region predisposed to somatic hypermutation, IgH FR1, IgH FR2 or IgH FR3.

In a further embodiment said contiguous nucleotide region corresponds to 75%, 76%, 77%, 78%, 79%, 80%, 81%, 82% or 83% of the maximum forward and reverse read length deliverable by the bidirectional sequencing technology selected for use in step (iii) and said forward and reverse read portions is not less than 75%, 76%, 77%, 78%, 79%, 80%, 81%, 82% or 83% of the maximum forward and reverse read length deliverable by the bidirectional sequencing technology selected for use in step (iii).

In yet another embodiment, said target DNA sequences are localised to the 120 contiguous nucleotides at the 5′ and/or 3′ terminal ends of said template but wherein the 20 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites.

In yet still another embodiment, said target DNA sequences are localised to the 125 contiguous nucleotides at the 5′ and/or 3′ terminal ends of said template but wherein up to the 30 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites.

In another further embodiment, said linker is 5-30 nucleotides in length, preferably 5-25 and more preferably 5-20. In another embodiment, the length of said linker is 5, 6, 7, 8, 9, 10, 11, 12, 13, 14, 15 or 16 nucleotides.

In still another further embodiment said analysis comprises aligning the nucleic acid sequence results generated in step (iv) and determining the expression of the target nucleic acid sequences of interest.

In a related aspect there is provided a method of diagnosing, monitoring or otherwise screening for a condition in a patient, which condition is characterised by the expression of one or more target nucleotide sequences, said method comprising:

(i) spatially isolating on a solid support a library of individual template DNA molecules derived from a nucleic acid sample, which template DNA molecules have been generated such that the target nucleotide sequences are localised to the region of contiguous nucleotides at the 5′ and/or 3′ terminal ends of said template;

(ii) amplifying said spatially isolated template DNA molecules to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read;
and wherein:
(1) said portion is not less than 75% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all the nucleic acid sequence results of (a) and the linker sequence is the same for all the nucleic acid sequence results of (b); and

(v) analysing the sequence result.

In one embodiment, said condition is characterised by a clonal population of cells or microorganisms.

In another embodiment, said clonal cells are a population of clonal lymphoid cells.

In still another embodiment, said condition is characterised by one or more target nucleotide sequences which are expressed by an immune cell.

In still yet another embodiment said contiguous nucleotide region of step (i) corresponds to about 80% of the maximum forward and reverse read length deliverable by the bidirectional sequencing technology selected for use in step (iii)

In yet still another embodiment said condition is characterised by the expression of one or more rearranged V, D or J gene segment sequence characteristics.

In another embodiment said DNA sample of interest comprises B and/or T cell DNA and said one or more target nucleotide sequences are one or more rearranged V, D or J gene segments.

In yet another embodiment said target nucleotide sequences are the DJ or VDJ rearrangements of IgH, TCR β or TCR δ or the VJ rearrangement of Igκ, Igλ, TCRα or TCRγ. In still another embodiment, said rearrangement is a kappa deleting element rearrangement.

In still yet another embodiment, said target nucleotide sequences are a V gene segment region, such as a region predisposed to undergoing hypermutation and/or a J gene segment region encoding a portion of the CDR3.

In yet still another embodiment, said target nucleotide sequences are the gene segment regions encoding all or some of the V leader sequence, the V region predisposed to somatic hypermutation, IgH FR1, IgH FR2 or IgH FR3.

In a further embodiment said contiguous nucleotide region corresponds to 75%, 76%, 77%, 78%, 79%, 80%, 81%, 82% or 83% of the maximum forward and reverse read length deliverable by the bidirectional sequencing technology selected for use in step (iii) and said forward and reverse read portions is not less than 75%, 76%, 77%, 78%, 79%, 80%, 81%, 82% or 83% of the maximum forward and reverse read length deliverable by the bidirectional sequencing technology selected for use in step (iii).

In yet another embodiment, said target DNA sequences are localised to the 120 contiguous nucleotides at the 5′ and/or 3′ terminal ends of said template but wherein the 20 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites.

In yet still another embodiment, said target DNA sequences are localised to the 125 contiguous nucleotides at the 5′ and/or 3′ terminal ends of said template but wherein up to the 30 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites.

In another embodiment, said linker is 5-25 nucleotides in length. In still another embodiment said linker is 5-20 nucleotides in length. In a further embodiment, the length of said linker is 5, 6, 7, 8, 9, 10, 11, 12, 13, 14, 15 or 16 nucleotides, most preferably 9, 10, 11 or 12 nucleotides in length.

In still another embodiment, said analysis comprises aligning the nucleic acid sequence results generated in step (iv) and determining the expression of the target nucleic acid sequences of interest.

In yet another embodiment, said condition which is characterised by the expression of one or more rearranged V, D or J gene segment sequence characteristics is infection, transplantation, autoimmunity, immunodeficiency, allergy, neoplasia or any other condition characterised by T or B cell clonal expansion.

Said method is useful in the context of diagnosis, prognosis, classification, prediction of disease risk, detection of recurrence of disease, immune surveillance or monitoring prophylactic or therapeutic efficacy.

Disease conditions suitable for analysis in the context of lymphoid neoplasias include acute lymphoblastic leukaemia, acute lymphocytic leukaemia, acute myeloid leukemia, acute promyelocytic leukemia, chronic lymphocytic leukaemia, chronic myeloid leukemia, myeloproliferative neoplasms, such as myeloma, systemic mastocytosis, lymphoma and hairy cell leukemia

In one particular aspect, the method of the present invention is used to detect minimum residual disease in the context of lymphoid neoplasia.

In another embodiment, non-neoplastic diseases characterised by clonal lymphoid expansion include infection, allergy, autoimmunity, transplant rejection, immunotherapy, polycythemia vera, myelodysplasia and leukocytosis, such as lymphocytic leucocytosis.

Another aspect of the disclosure is directed to a computer-implemented method for preparing nucleic acid sequence results for analysis from non-overlapping sequence reads. The method comprises identifying forward sequence reads and reverse sequence reads from sequence reads of a cluster of amplicons wherein the cluster is generated from an individual spatially isolated template DNA molecule, and each sequence read is generated by a selected bidirectional sequencing technology, and wherein the forward sequence reads and the reverse sequence reads do not overlap and do not provide a contiguous read across the full length of any amplicon; and linking the forward sequence reads with the reverse sequence reads resulting in a plurality of first nucleic acid sequence results, such that each forward sequence read is linked to a reverse sequence read and each reverse sequence read is linked to a forward sequence read through a first nucleic acid linker sequence, wherein each linking is achieved by: concatenating the first nucleic acid linker sequence between the 3′ end of a portion of the terminal 5′ contiguous nucleic acid sequence of a forward sequence read and the reverse complement of a portion of the terminal 5′ contiguous nucleic acid sequence of a reverse sequence read, thereby producing a first nucleic acid sequence result comprising the portion of the forward sequence read, the first nucleic acid linker sequence, and the reverse complement of the portion of the reverse sequence read in that order; wherein (1) the length of the portion from the forward sequence read is not less than 75% of the maximum read length deliverable by the selected bidirectional sequencing technology, the length of the portion from the reverse sequence read is not less than 75% of the maximum read length deliverable by the selected bidirectional sequencing technology; (2) the length of the portion from the reverse sequence read is the same for all reverse sequence reads which are analysed; (3) the length of the portion from the forward sequence read is the same for all forward sequence reads which are analysed but may be the same or different to the length of the portion from the reverse sequence read and (4) the first nucleic acid linker sequence is the same for all first nucleic acid sequence results.

In some embodiments, the computer-implemented method further comprises: linking the forward sequence reads with the reverse sequence reads resulting in a plurality of second nucleic acid sequence results, such that each forward sequence read is linked to a reverse sequence read and each reverse sequence read is linked to a forward sequence read through a second nucleic acid linker sequence, wherein each linking is achieved by concatenating the second nucleic acid linker sequence between the 3′ end of a portion of the terminal 5′ contiguous nucleic acid sequence of a reverse sequence read and the reverse complement of a portion of the terminal 5′ contiguous nucleic acid sequence of a forward sequence read, thereby producing a second nucleic acid sequence result comprising the portion from the reverse sequence read, the second nucleic acid linker sequence and the reverse complement of the portion from the forward sequence read in that order, wherein (1) the length of the portion from the forward sequence read is not less than 75% of the maximum read length deliverable by the selected bidirectional sequencing technology, the length of the portion from the reverse sequence read is not less than 75% of the maximum read length deliverable by the selected bidirectional sequencing technology; (2) the length of the portion from the reverse sequence read being concatenated to the second nucleic acid linker is the same for all reverse sequence reads and is the same as the length of the portion from the reverse sequence read being concatenated to the first nucleic acid linker, (3) the length of the portion from the forward sequence read being concatenated to the second nucleic acid linker is the same for all forward sequence reads and is the same as the length of the portion from the forward sequence read being concatenated to the first nucleic acid linker, but may be the same or different to the length of the portion from the reverse sequence read being concatenated to the second nucleic acid linker; and (4) the second nucleic acid linker sequence is the same for all second nucleic acid sequence results.

Another aspect of the disclosure is directed to a non-transitory computer-readable storage medium having program instructions embodied therewith, the program instructions executable by a processing element of a device to cause the device to implement a method for preparing nucleic acid sequence results for analysis from non-overlapping sequence reads by: identifying forward sequence reads and reverse sequence reads from sequence reads of a cluster of amplicons wherein the cluster is generated from an individual spatially isolated template DNA molecule, and each sequence read is generated by a selected bidirectional sequencing technology, and wherein the forward sequence reads and the reverse sequence reads do not overlap and do not provide a contiguous read across the full length of any amplicon; and linking the forward sequence reads with the reverse sequence reads resulting in a plurality of first nucleic acid sequence results, such that each forward sequence read is linked to a reverse sequence read and each reverse sequence read is linked to a forward sequence read through a first nucleic acid linker sequence, wherein each linking is achieved by: concatenating the first nucleic acid linker sequence between the 3′ end of a portion of the terminal 5′ contiguous nucleic acid sequence of a forward sequence read and the reverse complement of a portion of the terminal 5′ contiguous nucleic acid sequence of a reverse sequence read, thereby producing a first nucleic acid sequence result comprising the portion of the forward sequence read, the first nucleic acid linker sequence, and the reverse complement of the portion of the reverse sequence read in that order; wherein (1) the length of the portion from the forward sequence read is not less than 75% of the maximum read length deliverable by the selected bidirectional sequencing technology, the length of the portion from the reverse sequence read is not less than 75% of the maximum read length deliverable by the selected bidirectional sequencing technology; (2) the length of the portion from the reverse sequence read is the same for all reverse sequence reads which are analysed; (3) the length of the portion from the forward sequence read is the same for all forward sequence reads which are analysed but may be the same or different to the length of the portion from the reverse sequence read and (4) the first nucleic acid linker sequence is the same for all first nucleic acid sequence results.

In some embodiments, the non-transitory computer-readable storage medium further comprises linking the forward sequence reads with the reverse sequence reads resulting in a plurality of second nucleic acid sequence results, such that each forward sequence read is linked to a reverse sequence read and each reverse sequence read is linked to a forward sequence read through a second nucleic acid linker sequence, wherein each linking is achieved by concatenating the second nucleic acid linker sequence between the 3′ end of a portion of the terminal 5′ contiguous nucleic acid sequence of a reverse sequence read and the reverse complement of a portion of the terminal 5′ contiguous nucleic acid sequence of a forward sequence read, thereby producing a second nucleic acid sequence result comprising the portion from the reverse sequence read, the second nucleic acid linker sequence and the reverse complement of the portion from the forward sequence read in that order; wherein (1) the length of the portion from the forward sequence read is not less than 75% of the maximum read length deliverable by the selected bidirectional sequencing technology, the length of the portion from the reverse sequence read is not less than 75% of the maximum read length deliverable by the selected bidirectional sequencing technology; (2) the length of the portion from the reverse sequence read being concatenated to the second nucleic acid linker is the same for all reverse sequence reads and is the same as the length of the portion from the reverse sequence read being concatenated to the first nucleic acid linker, (3) the length of the portion from the forward sequence read being concatenated to the second nucleic acid linker is the same for all forward sequence reads and is the same as the length of the portion from the forward sequence read being concatenated to the first nucleic acid linker, but may be the same or different to the length of the portion from the reverse sequence read being concatenated to the second nucleic acid linker, and (4) the second nucleic acid linker sequence is the same for all second nucleic acid sequence results.

Another aspect of the disclosure is directed to a device for preparing nucleic acid sequence results for analysis from non-overlapping sequence reads. The device, comprises a hardware processor being configured to: identify forward sequence reads and reverse sequence reads from sequence reads of a cluster of amplicons wherein the cluster is generated from an individual spatially isolated template DNA molecule, and each sequence read is generated by a selected bidirectional sequencing technology, and wherein the forward sequence reads and the reverse sequence reads do not overlap and do not provide a contiguous read across the full length of any amplicon; an link the forward sequence reads with the reverse sequence reads resulting in a plurality of first nucleic acid sequence results, such that each forward sequence read is linked to a reverse sequence read and each reverse sequence read is linked to a forward sequence read through a first nucleic acid linker sequence, wherein each linking is achieved by: concatenating the first nucleic acid linker sequence between the 3′ end of a portion of the terminal 5′ contiguous nucleic acid sequence of a forward sequence read and the reverse complement of a portion of the terminal 5′ contiguous nucleic acid sequence of a reverse sequence read, thereby producing a first nucleic acid sequence result comprising the portion of the forward sequence read, the first nucleic acid linker sequence, and the reverse complement of the portion of the reverse sequence read in that order; wherein (1) the length of the portion from the forward sequence read is not less than 75% of the maximum read length deliverable by the selected bidirectional sequencing technology, the length of the portion from the reverse sequence read is not less than 75% of the maximum read length deliverable by the selected bidirectional sequencing technology; (2) the length of the portion from the reverse sequence read is the same for all reverse sequence reads which are analysed; (3) the length of the portion from the forward sequence read is the same for all forward sequence reads which are analysed but may be the same or different to the length of the portion from the reverse sequence read and (4) the first nucleic acid linker sequence is the same for all first nucleic acid sequence results.

In some embodiments, the hardware processor is further configured to link the forward sequence reads with the reverse sequence reads resulting in a plurality of second nucleic acid sequence results, such that each forward sequence read is linked to a reverse sequence read and each reverse sequence read is linked to a forward sequence read through a second nucleic acid linker sequence, wherein each linking is achieved by concatenating the second nucleic acid linker sequence between the 3′ end of a portion of the terminal 5′ contiguous nucleic acid sequence of a reverse sequence read and the reverse complement of a portion of the terminal 5′ contiguous nucleic acid sequence of a forward sequence read, thereby producing a second nucleic acid sequence result comprising the portion from the reverse sequence read, the second nucleic acid linker sequence and the reverse complement of the portion from the forward sequence read in that order, wherein (1) the length of the portion from the forward sequence read is not less than 75% of the maximum read length deliverable by the selected bidirectional sequencing technology, the length of the portion from the reverse sequence read is not less than 75% of the maximum read length deliverable by the selected bidirectional sequencing technology; (2) the length of the portion from the reverse sequence read being concatenated to the second nucleic acid linker is the same for all reverse sequence reads and is the same as the length of the portion from the reverse sequence read being concatenated to the first nucleic acid linker, (3) the length of the portion from the forward sequence read being concatenated to the second nucleic acid linker is the same for all forward sequence reads and is the same as the length of the portion from the forward sequence read being concatenated to the first nucleic acid linker, but may be the same or different to the length of the portion from the reverse sequence read being concatenated to the second nucleic acid linker; and (4) the second nucleic acid linker sequence is the same for all second nucleic acid sequence results.

In some embodiments, the first nucleic acid linker sequence and the second nucleic acid linker sequence are at least 11 nucleotides long.

In some embodiments, the length of the portion of the forward sequence read is the same as the length of the portion of the reverse sequence read.

In some embodiments, the portion of the forward sequence read comprises a specified number of contiguous nucleotides of the 5′ terminus of the forward sequence read, and the portion of the reverse sequence read comprises the specified number of contiguous nucleotides of the 5′ terminus of the reverse sequence read. In some embodiments, the specified number of contiguous nucleotides comprises between about 80 nucleotides and about 180 nucleotides.

In some embodiments, the forward and the reverse sequence reads are DNA sequence reads. In some embodiments, the cluster of amplicons is amplified from B and/or T cell DNA.

In some embodiments, the cluster of amplicons comprises at least one rearranged V, D or J gene segment.

### BRIEF DESCRIPTION OF THE DRAWINGS

FIG. 1. Block diagram of the system in accordance with the aspects of the disclosure. CPU: Central Processing Unit (“processor”).

FIG. 2. Flow chart of an embodiment for preparing nucleic acid sequence results for analysis from non-overlapping sequence reads.

FIG. 3. Flow chart of an embodiment for preparing nucleic acid sequence results for analysis from non-overlapping sequence reads.

### DETAILED DESCRIPTION OF THE INVENTION

The present invention is predicated, in part, on the development of a means to use non-overlapping bidirectional sequencing reads to screen for one or more target nucleotide sequences. Specifically, by virtue of the co-localisation of bidirectional sequence read results to an amplicon cluster which has been generated from a single template DNA anchored to a solid platform, and is therefore clonal, the sequencing information of those reads is identifiable as originating from a common template DNA. Methods to date have relied on the overlapping forward and reverse read sequences to enable assembly of the entire template DNA sequence from the bidirectional sequence reads or the use of a reference sequence against which the reads are aligned in order to determine their orientation and position relative to one another. This also provided the advantage that although sequencing errors are known to occur more frequently towards the 3′ terminal end of a sequence read, the overlapping complementary sequences of the paired read enabled identification of the presence of a single base error (as opposed to a mutation) on one strand, which could then be confidently discarded, and facilitated alignment and analysis of the taped reads to occur with relative accuracy. However, where bidirectional sequence reads do not overlap, their paring and assembly by virtue of overlapping complementary 3′ sequences is not possible. Still further, it has now been determined that even if the bidirectional sequence reads were to be individually analysed, aside from the problem of any sequencing errors which may have occurred at the 3′ end of the read and which would result in a single read being classified as a different (e.g. mutated) sequence relative to a comparative read which does not exhibit the error, the mere generation of different sequence read lengths, even if the actual sequences of these reads are otherwise identical, will result in these reads being incorrectly classified as different sequences, thereby skewing the sequencing results for the DNA sample of interest.

However, it has been unexpectedly determined that if the sequence reads are altered to cleave off sufficient of the 3′ bidirectional sequence read ends such that all sequence reads of the forward reads and reverse reads are of the same length, this unexpected phenomenon is rectified. Still further, if the forward and reverse reads are adjusted in this manner and then the 3′ ends of the forward and reverse reads, which are identified as being colocalised to a single amplicon cluster on the solid support, are linked using a nucleic acid linker which attaches to the 5′ ends of the of the sequences complementary to the reverse and forward reads, respectively, to generate a linear sequence read, and which linker is the same for all assembled reads for a given biological sample, an accurate alignment and comparative analysis of the assembled sequence results can be achieved. By designing the initial DNA template library such that the target nucleotide sequences are positioned at the 5′ and 3′ end of the template, and will therefore be sequenced by the selected bidirectional sequencing technology even if the entire template is not fully sequenced, there is provided a means to analyse potentially quite distantly positioned target nucleotide sequences, such as the VDJ gene segments which are rearranged in an immunoglobulin or TCR gene. By no longer being limited to choosing sequencing instrumentation based on the read length which it generates, rather than on other functional features of the instrumentation, and therefore being forced to design a template DNA library such that the template molecules are short enough to enable overlapping bidirectional sequence reads to be generated, there has now been enabled a wider application for high throughput next generation sequence analysis.

Accordingly, one aspect of the present invention is directed to a method of screening a nucleic acid sample of interest for the expression of one or more target nucleotide sequences, said method comprising:

(i) spatially isolating on a solid support a library of individual template DNA molecules derived from said nucleic acid sample, which template DNA molecules have been generated such that the target nucleotide sequences are localised to the region of contiguous nucleotides at the 5′ and/or 3′ terminal ends of said template;

(ii) amplifying said spatially isolated template DNA molecules to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read;
and wherein:
(1) said portion is not less than 75% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all the nucleic acid sequence results of (a) and the linker sequence is the same for all the nucleic acid sequence results of (b); and

(v) analysing the sequence result.

In one embodiment, said non-contiguous sequence reads are not analysed relative to a reference sequence in order to pair the forward and reverse reads.

Reference to a “nucleic acid” or “nucleotide” or “base” or “nucleobase” should be understood as a reference to both deoxyribonucleic acid or nucleotides and ribonucleic acid or nucleotides or purine or pyrimidine bases or derivatives or analogues thereof. In this regard, it should be understood to encompass phosphate esters of ribonucleotides and/or deoxyribonucleotides, including DNA (cDNA or genomic DNA), RNA or mRNA among others. The nucleic acid molecules of the present invention may be of any origin including naturally occurring (such as would be derived from a biological sample), recombinantly produced or synthetically produced. The nucleotide may also be a non-standard nucleotide such as inosine.

Reference to “derivatives” should be understood to include reference to fragments, parts, portions, homologs and mimetics of said nucleic acid molecules from natural, synthetic or recombinant sources. “Functional derivatives” should be understood as derivatives which exhibit any one or more of the functional activities of purine or pyrimidine bases, nucleotides or nucleic acid molecules. The derivatives of said nucleotides or nucleic acid sequences include fragments having particular regions of the nucleotide or nucleic acid molecule fused to other proteinaceous or non-proteinaceous molecules. The biotinylation of a nucleotide or nucleic acid molecules is an example of a “functional derivative” as herein defined. Derivatives of nucleic acid molecules may be derived from single or multiple nucleotide substitutions, deletions and/or additions. The term “functional derivatives” should also be understood to encompass nucleotides or nucleic acid exhibiting any one or more of the functional activities of a nucleotide or nucleic acid sequence, such as for example, products obtained following natural product screening.

“Analogs” contemplated herein include, but are not limited to, modifications to the nucleotide or nucleic acid molecule such as modifications to its chemical makeup or overall conformation or any other type of non-naturally occurring nucleotide. This includes, for example, modification to the manner in which nucleotides or nucleic acid molecules interact with other nucleotides or nucleic acid molecules such as at the level of backbone formation or complementary base pair hybridisation. Without limiting the present invention to any one theory or mode of action, nucleic acids are composed of three parts: a phosphate backbone, a pentose sugar, either ribose or deoxyribose and one of four bases. An analogue may have any of these altered. Typically the analogue bases confer, among other things, different base pairing and base stacking properties. Examples include universal bases, which can pair with all four canonical bases, and phosphate-sugar backbone analogues such as PNA, which affect the properties of the chain. Nucleic acid analogues are also called xeno nucleic acids. Non-naturally occurring nucleic acids include peptide nucleic acid (PNA), morpholino and locked nucleic acid (LNA), as well as glycol nucleic acid (GNA) and threose nucleic acid (TNA). Each of these is distinguished from naturally occurring DNA or RNA by changes to the backbone of the molecule.

The nucleic acid sample of interest and/or the target nucleotide sequence may be DNA or RNA or derivative or analogue thereof. Said nucleic acid sample may take the form of genomic DNA, cDNA which has been generated from an mRNA transcript, DNA generated by nucleic acid amplification, synthetic DNA or recombinantly generated DNA. If the subject nucleic acid sample is RNA, it would be appreciated that it will first be necessary to reverse transcribe the RNA to DNA, such as using RT-PCR. The subject RNA may be any form of RNA, such as mRNA, primary RNA transcript, ribosomal RNA, transfer RNA, micro RNA or the like. Preferably, said nucleic acid sample and said target nucleotide sequence is DNA.

According to this embodiment, there is provided a method of screening a DNA sample of interest for the expression of one or more target DNA sequences, said method comprising:

(i) spatially isolating on a solid support a library of individual template DNA molecules derived from said DNA sample, which template DNA molecules have been generated such that the target DNA sequences are localised to the region of contiguous nucleotides at the 5′ and/or 3′ terminal ends of said template;

(ii) amplifying said spatially isolated template DNA molecules to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read;
and wherein:
(1) said portion is not less than 75% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all the nucleic acid sequence results of (a) and the linker sequence is the same for all the nucleic acid sequence results of (b); and

(v) analysing the sequence result.

In one embodiment, said contiguous nucleotide region of step (i) corresponds to about 80% of the maximum forward and reverse read length deliverable by the bidirectional sequencing technology selected for use in step (iii).

Reference to a “target nucleotide sequence” should be understood as a reference to any DNA or RNA sequence which is sought to be analysed. This may be a gene, part of a gene, such as a gene segment or gene region, or an intergenic region. To this end, reference to “gene” should be understood as a reference to a DNA molecule which codes for a protein product, whether that be a full length protein or a protein fragment. In terms of chromosomal DNA, the gene will include both intron and exon regions. However, to the extent that the nucleic acid sample is cDNA, such as might occur if the target nucleotide sequence is vector DNA or reverse transcribed mRNA, there may not exist intron regions. Such DNA may nevertheless include 5′ or 3′ untranslated regions. Accordingly, reference to “gene” herein should be understood to encompass any form of DNA which codes for a protein or protein fragment including, for example, genomic DNA and cDNA. The subject target nucleotide sequence may also correspond to a non-coding portion of genomic DNA which is not known to be associated with any specific gene (such as the commonly termed “junk” DNA regions). It may correspond to any region of genomic DNA produced by recombination, either between two regions of genomic DNA or a region of genomic DNA and a region of foreign DNA such as a virus or an introduced sequence. It may also correspond to a region which may encompass a SNP, chromosomal translocation, insertion, deletion or breakpoint, such as a chromosomal breakpoint. The target sequence may also correspond to a region of a partly or wholly synthetically or recombinantly generated nucleic acid molecule. The subject target sequence may also be a region of DNA which has been previously amplified by any nucleic acid amplification method, including polymerase chain reaction (PCR) (i.e. it has been generated by an amplification method).

The method of the present invention is designed to screen for the “expression” of said one or more target nucleotide sequences. By “expression” is meant the presence of said sequence in the nucleic acid sample undergoing testing. It should be understood that the subject sequence may or may not correspond to a nucleic acid sequence which undergoes transcription and/or translation.

That the method of the present invention may be designed to screen for “one or more” target nucleotide sequences of interest should be understood to mean that one may screen for one or more than one distinct target sequence. Examples of distinct target sequences include a SNP, point mutation, hypermutation, DNA insertion, DNA deletion, chromosomal breakpoint, a specific gene segment, a specific region, part or section of a gene, intergenic region or the like. One may screen for one of these target sequences or one may screen for more than one of these target sequences in the context of a single analysis. These target sequences may be located at separate and distinct positions in the nucleic acid of the sample or they may be located sequentially along a nucleic acid strand. It should be understood that they may even occur in the same position along a nucleic acid strand, such as where a mutation is found within a gene segment and wherein both the mutation and the gene segment itself are target sequences of interest. In one embodiment said nucleic sample of interest comprises B and/or T cell DNA and said one or more target nucleotide sequences are one or more rearranged V, D or J gene segments.

In accordance with this embodiment there is provided a method of screening a DNA sample comprising B and/or T cell DNA for the expression of one or more rearranged V, D or J gene segments, said method comprising:

(i) spatially isolating on a solid support a library of individual template DNA molecules derived from said DNA sample, which template DNA molecules have been generated such that said rearranged V, D or J gene segments are localised to the region of contiguous nucleotides at the 5′ and/or 3′ terminal ends of said template;

(ii) amplifying said spatially isolated template DNA molecules to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read:
and wherein:
(1) said portion is not less than 75% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all the nucleic acid sequence results of (a) and the linker sequence is the same for all the nucleic acid sequence results of (b); and

(v) analysing the sequence result.

It should be understood that reference to “B and/or T cell DNA” is a reference to DNA derived from any lymphoid cell which has rearranged at least one germ line set of immunoglobulin or TCR variable region gene segments. The immunoglobulin variable region encoding genomic DNA which may be rearranged includes the variable regions associated with the heavy chain or the κ or λ light chain while the TCR chain variable region encoding genomic DNA which may be rearranged include the α, β, γ and δ chains. In this regard, a cell should be understood to fall within the scope of “lymphoid cell” provided the cell has rearranged the variable region encoding DNA of at least one immunoglobulin or TCR gene segment region. It is not necessary that the cell is also transcribing and translating the rearranged DNA. In this regard, “lymphoid cell” includes within its scope, but is in no way limited to, immature T and B cells which have rearranged the TCR or immunoglobulin variable region gene segments but which are not yet expressing the rearranged chain (such as TCR thymocytes) or which have not yet rearranged both chains of their TCR or immunoglobulin variable region gene segments. This definition further extends to lymphoid-like cells which have undergone at least some TCR or immunoglobulin variable region rearrangement but which cell may not otherwise exhibit all the phenotypic or functional characteristics traditionally associated with a mature T cell or B cell.

It should also be understood that although in one embodiment the subject rearrangement is a completed rearrangement, such as the completed rearrangement of at least one variable region gene region, in another embodiment the subject rearrangement is a partial rearrangement. For example, a B cell which has only undergone the DJ recombination event is a cell which has undergone only partial rearrangement. Complete rearrangement will not be achieved until the DJ recombination segment has further recombined with a V segment. The method of the present invention can therefore be designed to screen the partial or complete variable region rearrangement of the TCR or immunoglobulin chain.

Without limiting the present invention to any one theory or mode of action, V(D)J recombination in organisms with an adaptive immune system is an example of a type of site-specific genetic recombination that helps immune cells rapidly diversify to recognise and adapt to new pathogens. Each lymphoid cell undergoes somatic recombination of its germ line variable region gene segments (either V and J, D and J or V, D and J segments), depending on the particular gene segments rearranged, in order to generate a total antigen diversity of approximately 10¹⁶ distinct variable region structures. In any given lymphoid cell, such as a T cell or B cell, at least two distinct variable region gene segment rearrangements are likely to occur due to the rearrangement of two or more of the two chains comprising the TCR or immunoglobulin molecule, specifically, the α, β, γ or δ chains of the TCR and/or the heavy and light chains of the immunoglobulin molecule. In addition to rearrangements of the VJ, DJ or VDJ segment of any given immunoglobulin or TCR gene, nucleotides are randomly removed and/or inserted at the junction between the segments. This leads to the generation of enormous diversity.

The loci for these gene segments are widely separated in the germline but recombination during lymphoid development results in apposition of a V, (D) and J gene, with the junctions between these genes being characterised by small regions of insertion and deletion of nucleotides. This process occurs randomly so that each normal lymphocyte comes to bear a unique V(D)J rearrangement. Since a lymphoid cancer, such as acute lymphoblastic leukaemia, chronic lymphocytic leukaemia, lymphoma or myeloma, occurs as the result of neoplastic change in a single normal cell, all of the cancer cells will, at least originally, bear the junctional V(D)J rearrangement originally present in the founder cell. Subclones may arise during expansion of the neoplastic population and further V(D)J rearrangements may occur in them.

Reference to a “gene segment” should be understood as a reference to the V, D and J regions of the immunoglobulin and T cell receptor genes. The V, D and J gene segments are clustered into families. For example, there are 52 different functional V gene segments for the κ immunoglobulin light chain and 5 J gene segments. For the immunoglobulin heavy chain, there are 55 functional V gene segments, 23 functional D gene segments and 6 J gene segments. Across the totality of the immunoglobulin and T cell receptor V, D and J gene segment families, there are a large number of individual gene segments, thereby enabling enormous diversity in terms of the unique combination of V(D)J rearrangements which can be affected. For the sake of clarity, the rearranged immunoglobulin or T cell receptor [V(D)J] variable nucleic acid region will be referred to herein as a rearranged “gene” and the individual V, D or J nucleic acid regions will be referred to as “gene segments”. Accordingly, the terminology “gene segment” is not exclusively a reference to a segment of a gene. Rather, in the context of Ig and TCR gene rearrangement, it is a reference to a gene in its own right with these gene segments being clustered into families. A “rearranged” immunoglobulin or T cell receptor variable region gene should be understood herein as a gene in which two or more of one V segment, one J segment and one D segment (if a D segment is incorporated into the particular rearranged variable gene in issue) have been spliced together to form a single rearranged “gene”. In fact, this rearranged “gene” is actually a stretch of genomic DNA comprising one V gene segment, one J gene segment and one D gene segment which have been spliced together. It is therefore sometimes also referred to as a “gene region” since it is actually made up of 2 or 3 distinct V, D or J genes (herein referred to as gene segments) which have been spliced together. The individual “gene segments” of the rearranged immunoglobulin or T cell receptor gene are therefore defined as the individual V, D and J genes. These genes are discussed in detail on the IMGT database. The term “gene” will be used herein to refer to the rearranged immunoglobulin or T cell receptor variable gene. The term “gene segment” will be used herein to refer to the V, D and J segments. However, it should be noted that there is significant inconsistency in the use of “gene”/“gene segment” language in terms of immunoglobulin and T cell receptor rearrangement. For example, the IMGT refers to individual V, D and J “genes”, while some scientific publication refers to these as “gene segments”. Some sources refer to the rearranged variable immunoglobulin or T cell receptor as a “gene region” while others refer to it as a “gene”. The nomenclature which is used in this specification is as defined earlier.

Still without limiting the present invention to any one theory or mode of action, the nature of genetic recombination events is such that a junction between the recombined genes or gene segments (as defined herein) may be characterised by the deletion and insertion of random nucleotides resulting in the formation of “N regions”. These N regions are also unique and are themselves sometimes therefore useful targets in the context of target sequence analysis. Accordingly, it is generally understood that the V(D)J rearrangement provides combinatorial diversity while the addition of N nucleotides or palindromic (P) nucleotides provides junctional diversity.

It should also be understood that within the context of V(D)J rearrangement, the secondary structure of the protein molecule which is translated does itself comprise unique features which are themselves often the subject of analysis, albeit it in terms of the DNA sequence regions within the V(D)J rearrangement which encode these secondary structure features. For example, the translated variable region of IgH (the immunoglobulin heavy chain) or the TCR β or S chains takes the form of three looped hypervariable regions which are usually referred to as the complementary determining regions (CDR) 1, 2 and 3. These CDR regions are flanked by four framework regions (FR) 1, 2, 3 and 4. Without limiting the present invention to any one theory or mode of action, the V gene segment is understood to encode the CDR1, CDR2, leader sequence, FR1, FR2 and FR3. The CDR3 region is encoded by part of the V gene segment, all of the D gene segment and part of the J gene segment. The remainder of the J gene segment generally encodes FR4.

Accordingly, in one embodiment and in the context of V(D)J rearrangement, said target nucleotide sequences are the DJ or VDJ rearrangements of IgH, TCR β or TCR δ. In another embodiment said target nucleotide sequences are the VJ rearrangement of Igκ, Igλ, TCRα or TCRγ. In yet another embodiment, said rearrangement is a kappa deleting element rearrangement.

In yet another embodiment, said target nucleotide sequences are a V gene segment region, such as a region predisposed to undergoing hypermutation and/or a J gene segment region encoding a portion of the CDR3.

In still yet another embodiment, said target nucleotide sequences are the gene segment regions encoding all or some of the V leader sequence, the V region predisposed to somatic hypermutation, IgH FR1, IgH FR2 or IgH FR3.

In yet still another embodiment, said target nucleotide sequence is the BCL1/JH or BCL2/JH t(14:18) translocations.

In still yet another embodiment, said target nucleotide sequence is an internal tandem duplication or other mutation associated with the FLT3 or TP53 genes.

In terms of the nature of the target nucleotide sequence, the method of the present invention facilitates screening either for the presence of a specific nucleotide sequence, such as a specific V, D or J gene segment sequence or screening a target nucleotide sequence region to determine the diversity of sequences expressed by the DNA molecules of that region. In this example, the target nucleotide sequence might be a V, D or J gene segment family, rather than a specific V, D or J gene segment, thereby enabling determination of the nature and diversity of gene segments within that family which are expressed by the DNA sample of interest.

The method of the present invention provides a significant improvement to traditional solid phase next generation sequencing techniques which are based on the use of cluster amplification of individual template sequences followed by bidirectional sequencing. Without limiting the present invention to any one theory or mode of action, in one embodiment of this type of technology, subsequently to the preparation of a library of DNA templates for analysis, these templates are anchored to a solid support via an adaptor sequence. Once attached, cluster generation can begin. The objective is to create hundreds of identical strands of the template DNA. Some will correspond to the forward strand and others to the complementary reverse strand. Clusters are then generated through bridge amplification. Polymerases move along a strand of DNA, generating its complementary strand. The original strand is washed away, leaving only the reverse strand. At the top of the reverse strand there is another adaptor sequence. The DNA strand bends and attaches to an anchored oligonucleotide that is complementary to this adaptor sequence. Polymerases then attach to the reverse strand, and its complementary strand (which is identical to the original strand) is generated. The now double stranded DNA is denatured so that each strand can separately attach to other unoccupied anchored oligonucleotide sequences which are complementary to the adaptors present at each end of the amplicons. This bridge amplification proceeds to simultaneously generate thousands of clusters corresponding to individual templates across the solid support (often referred to as a “flow cell”). The amplification is therefore clonal within the context of an individual cluster since each cluster is generated from a single starting template DNA.

Subsequently to clonal amplification, the reverse strands are washed off the flow cell, leaving only forward strands. Sequencing by synthesis using reversibly terminated fluorescently labelled oligonucleotides then commences. Primer, attach to the forward strands and a polymerase adds fluorescently tagged nucleotides to the DNA strand. Only one base is added per round. A reversible terminator which is present on every nucleotide prevents multiple additions in one round. Each of the four bases produces a unique emission, and after each round, the instrumentation which is used records which base was added based on the emitted fluorescence. Once the forward DNA strand has been read and the sequence read washed away, the reverse strand is generated via another round of bridge amplification. The forward strand is then washed away and the process of sequence by synthesis repeats for the reverse strand. In this way, bidirectional sequencing is achieved.

The present invention improves on this method by virtue of the design of a means to generate and correctly pair and assemble non-overlapping bidirectional sequence reads of a DNA template which is longer than the selected bidirectional sequence read length. This is achieved, in part, by the unique design of the library of template DNA molecules which are derived from the nucleic acid sample. Reference to a “template” DNA molecule in this regard should be understood as a reference to the DNA molecule which is to be anchored to a solid support (“spatially isolated”) and thereafter amplified to generate a cluster of clonal amplicons. That is, this molecule comprises both the target nucleic acid region and any additional nucleic acid or non-nucleic acid regions hereinafter described in more detail (such as nucleic acid adaptor sequences, sequencing primer hybridisation regions, index regions, unique molecular identifiers and the like. In this regard, the template DNA molecule which undergoes cluster amplification and sequencing is a single stranded molecule but it should be understood that at the time of anchoring to the solid support the DNA template may be either in single stranded form or it may form part a molecular complex, such as a double stranded DNA molecule or a complex with a non-nucleic acid component. For example, it may be desirable to enrich the template population prior to anchoring and this may be achieved by coupling a bead or chemical compound (e.g. biotin) to the particular template DNA molecules of interest in order to enable their isolation and thereby enrichment prior to anchoring. However, to the extent that a double stranded or other molecular complex is anchored, the skilled person would appreciate that the complex will have to be rendered single stranded prior to cluster amplification such that only the anchored template DNA is amplified. In this regard, it is envisaged that to the extent that the template DNA is coupled to a non-nucleic acid molecule which will not interfere with amplification, such as biotin, this non-nucleic acid molecule need not necessarily be cleaved off. The reference to “template” DNA molecule is therefore intended as a reference to the DNA molecule which will actually undergo amplification. By “library” of template DNA is meant the population of template DNA molecules (in single stranded, double stranded or some other complexed form) which are initially applied and anchored to the solid support. It should be understood that the template DNA may be comprised of either naturally or non-naturally occurring nucleotides, as hereinbefore described.

The template DNA molecules which are applied to the solid support are “derived from” the nucleic acid sample of interest. By “derived from” is meant that the template DNA is either directly isolated from the sample, as would occur if the DNA of the sample is simply fragmented prior to application to the solid support, or it takes the form of an amplification product which is generated from the DNA sample of interest. In this regard, the template DNA library can be prepared using any suitable method. The library maybe generated by fragmentation of the nucleic acid sample of interest, such as by using endonucleases, in particular restriction enzymes, exonucleases, exo-endonucleases or any other means of site directed DNA cleavage. Depending on the nature and location of the target nucleotide sequences, this method may be sufficient to generate a library. Alternatively, to facilitate enrichment of the target nucleotide sequence, one may elect to amplify the sample of interest using primers which will specifically target and amplify the nucleotide sequence of interest, for example primers directed to amplifying specific immunoglobulin or TCR gene segment rearrangements, primers which amplify gene regions that may have developed SNPs or primers which amplify across specific indels, breakpoints or other chromosomal translocations or mutations. The template DNA molecule may be of any suitable length, for example, 250-1000, 250-900, 300-700 or 300-600 nucleotides in length. It would be appreciated by the person of skill in the art that the portion of the template DNA molecule which corresponds to the target nucleic acid region will generally be smaller than the length of the template DNA since the template DNA may also incorporate adaptor regions and the like which will facilitate solid phase amplification and sequencing. In this regard, these additional non-target regions may comprise 15-75 nucleotides at each end of a template DNA molecule, preferably 20-40 and more preferably 20, 21, 22, 23, 24, 25, 26, 27, 28, 29 or 30 nucleotides in length.

Regardless of whether the template DNA molecules take the form of fragmented DNA or are amplified from all or some of the DNA sample of interest, said template DNA may also undergo further modification to introduce additional nucleic acid or non-nucleic acid components which are necessary or desirable to facilitate the efficacy of the high throughput amplification and sequencing platform technology which is used in the context of the present invention. Such additional sequences, include, for example, restriction enzyme sites or certain nucleic acid tags to enable amplification products of a given nucleic acid template sequence to be identified. Other desirable sequences include fold-back DNA sequences (which form hairpin loops or other secondary structures when rendered single-stranded), ‘control’ DNA sequences which direct protein/DNA interactions, such as for example a promoter DNA sequence which is recognised by a nucleic acid polymerase or an operator DNA sequence which is recognised by a DNA-binding protein. In another example, in order to enable anchoring of the template DNA to the solid support, a means for attaching the template DNA to the solid support is required to be coupled to the template DNA. In this regard, “means for attaching the template DNA to a solid support” as used herein refers to any chemical or non-chemical attachment method including chemically-modifiable functional groups. “Attachment” relates to immobilization of template DNA on a solid support by either a covalent or non-covalent attachment including via irreversible passive adsorption or via affinity between molecules (for example, immobilization on an avidin-coated surface by biotinylated molecules) or hybridization (such as between short complementary nucleic acid fragments). The attachment must be of sufficient strength that it cannot be removed by washing with water or aqueous buffer under DNA-denaturing conditions. “Chemically-modifiable functional group” as used herein refers to a group such as for example, a phosphate group, a carboxylic or aldehyde moiety, a thiol, or an amino group. To this end, reference to a “solid support” should be understood as reference to any solid surface to which nucleic acids can be covalently attached, such as for example latex beads, dextran beads, polystyrene, polypropylene surface, polyacrylamide gel, gold surfaces, glass surfaces and silicon wafers. Means for selecting a suitable solid support and attaching the template DNA would be well known to the person of skill in the art. In one embodiment, said solid support is a solid matrix whose two dimensional position can be ascertained. In another embodiment, said solid support is a glass surface (such as a glass slide or flow cell) and said means for anchoring the template to the glass surface is a nucleic acid anchor.

According to this embodiment, there is provided a method of screening a DNA sample of interest for the expression of one or more target DNA sequences, said method comprising:

(i) spatially isolating on a glass surface a library of individual template DNA molecules derived from said DNA sample, which template DNA molecules have been generated such that the target DNA sequences are localised to the region of contiguous nucleotides at the 5′ and/or 3′ terminal ends of said template;

(ii) amplifying said spatially isolated template DNA molecules to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read:
and wherein:
(1) said portion is not less than 75% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all the nucleic acid sequence results of (a) and the linker sequence is the same for all the nucleic acid sequence results of (b); and

(v) analysing the sequence result.

Preferably said glass surface is a glass slide or a flow cell.

In another embodiment said nucleic sample of interest comprises B and/or T cell DNA and said one or more target nucleotide sequences are one or more rearranged V, D or J gene segments.

In yet another embodiment said target nucleotide sequences are the DJ or VDJ rearrangements of IgH, TCR β or TCR δ or the VJ rearrangement of Igκ, Igλ, TCRα or TCRγ. In another embodiment, said rearrangement is a kappa deleting element rearrangement.

In still yet another embodiment, said target nucleotide sequences are a V gene segment region, such as a region predisposed to undergoing hypermutation and/or a J gene segment region encoding a portion of the CDR3.

In yet still another embodiment, said target nucleotide sequences are the gene segment regions encoding all or some of the V leader sequence, the V region predisposed to somatic hypermutation, IgH FR1, IgH FR2 or IgH FR3.

A typical example of a nucleic acid anchoring system is a short linear nucleic acid sequence (herein referred to as a “nucleic acid adaptor”) which is attached to the terminal 5′ and/or 3′ ends of the template DNA molecule. The anchor takes the form of a complementary nucleic acid sequence which is covalently bound to the solid support. Once the template DNA is applied to the solid support, any nucleic acid adaptor sequences which are complementary to the covalently bound nucleic acid anchors will result in hybridization of the two sequences and, thereby, anchoring of the template DNA to the solid support. In this regard, the 5′ nucleic acid adaptor sequence which is attached to a template DNA may be designed to express the same sequence as that of the corresponding anchor sequence, such that only the complementary sequence to the 5′ adaptor will hybridize to the anchor, while the 3′ nucleic acid adaptor sequence is complementary to its corresponding anchor. In this way, as the full length of the template DNA sequence undergoes cluster amplification, hybridization of the adaptor sequences on the 3′ end of the DNA template to the corresponding anchor, amplification of the amplicons generated from the DNA template is constantly facilitated, thereby enabling bridge amplification and cluster formation to constantly occur. As would be appreciated by the skilled person, this is the principle upon which the Illumina MiSeq, HiSeq, NovaSeq, and NextSeq instrumentation, for example, operates.

Reference to “spatially isolating” the individual template DNA molecules on a solid support should therefore be understood as a reference to anchoring these molecules to the solid support in order to enable cluster amplification of the templates. To this end, said template molecules are “spatially” isolated provided that the concentration of molecules applied to the solid support is such that the distribution and anchoring of these molecules across the solid support leaves sufficient unoccupied anchor molecules proximal to each anchored template DNA molecule so that localised clonal cluster amplification can occur without the amplicons of any one clonal cluster merging substantially into another cluster, thereby enabling bidirectional sequencing data from a single template to paired, with a high degree of accuracy, based on co-localisation data. That is, the amplicons of a single cluster are maintained within a discrete area on the solid support, and cluster density optimized so that data can be spatially assigned. In this regard, it is well within the skill of the person in the art to determine optimal cluster density for the instrumentation which is selected for use As would be appreciated by the person of skill in the art, each cluster may comprise both the forward strand and the complementary reverse strand for each initial template DNA molecule.

In addition to the adaptor molecule which may be incorporated into the template DNA molecule to facilitate anchoring of the template DNA to the solid support, the template DNA molecule may also be modified to incorporate additional features which are useful in a clinical or research setting, such as indexes, barcodes, unique molecular identifiers, sequencing primer hybridisation sites, index sequencing primer hybridisation sites and the like. For example, one may design the template DNA molecule such that in addition to localising the target nucleotide sequences of interest to the 5′ and 3′ ends of the template as hereinbefore described, the template is modified to incorporate an additional nucleic acid sequence region which is (a) adjacent to target nucleotide sequence region and (b) positioned at the terminal ends of either or both of the 5′ and 3′ ends of the template DNA molecule, together with the adaptor. This additional nucleic acid sequence region therefore expresses one or more of an adaptor sequence, a demultiplexing index (also commonly referred to as a barcode) such that multiple different nucleic acid samples can be simultaneously analysed, a unique molecular identifier to enable identification of individual amplicons, a sequencing primer hybridisation site and an index sequencing primer hybridisation site. The combination of features which are selected to be incorporate at the 5′ end of the template DNA need not be the same as those which are incorporated at the 3′ end. For example, a demultiplexing index may only be incorporated at one end of the template DNA strand. It is well within the skill of the person in the art to design such additional features into a template DNA in order to facilitate an optimal experimental design. Means for incorporating such additional nucleic acid components are well known and include blunt end ligation of a nucleic acid fragment comprising these features to the 5′ and/or 3′ ends of the template DNA molecule. Alternatively, if the template library is prepared by amplifying the DNA of the sample of interest, for example by PCR, one may design the amplification primers to include these additional features at their 5′ terminal ends. In this way, the primers which have been designed to amplify the target nucleotide sequences of interest can be designed to simultaneously incorporate these additional nucleic acid sequences, thereby generating the library in a single amplification step. In another alternative, one may elect to use a two-step amplification procedure to prepare the library wherein in the first round amplification primers directed to generating the template DNA amplicons expressing the target nucleotide sequences are used followed by primers directed to all amplicons generated from the first round (eg. consensus primers), which primers achieve the incorporation of exogenous DNA such as the indexes and the like discussed earlier.

In one embodiment, said template DNA molecule additionally expresses one or more nucleic acid sequences corresponding to indexes, barcodes, unique molecular identifiers, sequencing primer hybridisation sites and index sequencing primer hybridisation sites at the terminal 5′ and/or 3′ position.

According to this embodiment, there is provided a method of screening a DNA sample of interest for the expression of one or more target DNA sequences, said method comprising:

(i) spatially isolating on a glass surface a library of individual template DNA molecules derived from said DNA sample, which template DNA molecules have been generated such that the target DNA sequences are localised to the region of contiguous nucleotides at the 5′ and/or 3′ terminal ends of said template, wherein the terminal end of said contiguous nucleotide region expresses one or more nucleic acid sequences corresponding to indexes, barcodes, unique molecular identifiers, sequencing primer hybridisation sites and index sequencing primer hybridisation sites;

(ii) amplifying said spatially isolated template DNA molecules to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read;
and wherein:
(1) said portion is not less than 75% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all the nucleic acid sequence results of (a) and the linker sequence is the same for all the nucleic acid sequence results of (b); and

(v) analysing the sequence result.

Preferably said glass surface is a glass slide or a flow cell.

In another embodiment said nucleic sample of interest comprises B and/or T cell DNA and said one or more target nucleotide sequences are one or more rearranged V, D or J gene segments.

In yet another embodiment said target nucleotide sequences are the DJ or VDJ rearrangements of IgH, TCR β or TCR δ or the VJ rearrangement of Igκ, Igλ, TCRα or TCRγ. In still another embodiment, said rearrangement is a kappa deleting element rearrangement.

In still yet another embodiment, said target nucleotide sequences are a V gene segment region, such as a region predisposed to undergoing hypermutation and/or a J gene segment region encoding a portion of the CDR3.

In yet still another embodiment, said target nucleotide sequences are the gene segment regions encoding all or some of the V leader sequence, the V region predisposed to somatic hypermutation, IgH FR1, IgH FR2 or IgH FR3.

As detailed hereinbefore, the present invention has facilitated the routine use of high throughput bidirectional sequencing even where the template DNA is longer than what the bidirectional sequencing chemistry can read. However, this development is based, in part, on the design of the template DNA molecules such that the target nucleotide sequences are located within the region of contiguous nucleotides at the 5′ and/or 3′ terminal ends of the template. More specifically, the target sequences should be located within the stretch of 5′ and/or 3′ terminal nucleotides which correspond to about 80% of the maximum read length which is deliverable by the bidirectional sequencing technology which is selected for use. In this regard, reference to “bidirectional sequencing” (also commonly referred to as paired-end sequencing) should be understood as a reference to obtaining sequence information in relation to a template DNA molecule from both its 5′ and 3′ ends. In practice, this is achieved by sequencing the template DNA which has been amplified by cluster formation on the solid support. Sequencing of the strand which is complementary to the target strand (also known as the “template strand” or “template amplicon”) from its 3′ end produces the “reverse read”. The sequence of this read is complementary to the target strand. Sequencing of the complement to the target strand from the 3′ end of this complementary strand produces the “forward read”. The sequence of this read corresponds to the template strand. The two reads are therefore the reverse complements of the 100 or so (depending on the sequencing chemistry which is used) most 3′ nucleotides of the template strand and its complementary strand.

Where the template strand is shorter than the combined forward and reverse bidirectional sequence read lengths, the forward and reverse reads will overlap and exhibit complementarity in the overlapped regions. Based on these reads, the full length sequence of the template strand and its complement can be inferred. However, this is not possible where the template strand is longer than the combined read lengths of the bidirectional forward and reverse reads since the central region of the template strand will not have been sequenced by either of the reads. As discussed herein, the method of the present invention has provided an improved means of performing high throughput bidirectional sequencing such that its application can be extended to any template DNA molecule (and therefore its template strand amplicon), irrespective of its length.

The sample of the present invention comprises both the strand which expresses the target nucleotide sequence and the opposite strand of the target nucleotide sequence of interest DNA comprises two complimentary strands of DNA which hybridise together to form a molecule. The target nucleotide sequence which is the subject of interest is defined, in the context of the present invention, as the “forward strand” (also the “template strand” or “target strand”) while the complementary strand is referred to as the “reverse strand”. The skilled person would appreciate that the two strands of a DNA double helix are also often referred to as the “sense” strand, “coding” strand, “positive (+)” strand, “top” strand or “upper” strand. These latter three terms are more commonly utilised where the DNA region of interest does not produce a protein expression product. The corresponding complementary strand is often referred to as the “antisense” strand, non-coding” strand, “negative (−)” strand, “lower” strand or “bottom” strand. This should be understood to mean the strand which, in the context of the chromosomal locus, is complementary to the top/+/upper strand and, in its natural state, hybridizes to the top strand to form the characteristic double helix structure. As would be appreciated by the person of skill in the art, this nomenclature has become progressively less precise as it has been determined that there are many gene regions that do not code for proteins (and are not therefore correctly described as being found on the sense or coding strand) and, further, that genes may be found on either the +/upper strand or the −/lower strand, depending on how the skilled person defines these strands. Even genes which code for proteins are now known to be found on what was traditionally regarded as the −/bottom/antisense strand. Accordingly, identifying and defining a strand by reference to this terminology alone, and without reference to a specific chromosomal position or by reference to the specific +/− strand nomenclature used in the annotated human genome data base, may be imprecise. In this regard, in the context of the present invention a reference to the “forward strand” is a reference to the DNA strand which comprises the nucleotide sequence of interest, whichever of the two strands this is, while the “reverse strand” is a reference to the complementary strand. The target strand may therefore correspond to either the +/− (top/bottom, upper/lower) strand in the original DNA biological sample, depending on where the gene is positioned on the chromosomal double helix. “Forward strand” and “reverse strand” should be distinguished from the definitions of “forward read” and “reverse read” as hereinbefore described.

As detailed hereinbefore, the DNA template which is derived from the nucleic acid sample is designed such that the one or more target nucleotide sequences of interest are localised to the 5′ and/or 3′ terminal ends of the template. In this regard, reference to the “terminal end” of the DNA template is a reference to the region of nucleic acid sequence which runs contiguously from the most terminal 5′ nucleotide in the 3′ direction along the template strand and which runs from the most terminal 3′ nucleotide in the 5′ direction along the template strand. More specifically, the target nucleotide sequence is located within the contiguous stretch of nucleotides which run from the terminal 5′ and/or 3′ nucleotide, in the 3′ and 5′ direction respectively, for a contiguous number of nucleotides equivalent to about 80% of the maximum forward or reverse read length which is deliverable by the bidirectional sequencing technology which is selected for use. Reference to “the forward and reverse read length” should be understood as a reference to the read length of a single read and not the combined length of both reads. For example, the Illumina NovaSeq 6000 instrumentation will enable a maximum cycle run of 300, which equates to a bidirectional sequencing read length of 150 nucleotides for the forward read and 150 nucleotides for the reverse read, 80% of which would be 105 nucleotides per read. Reference to the “maximum read length” is therefore a reference to the maximum read length for either the forward read or the reverse read (eg. 150 for NovaSeq 6000) which the selected instrumentation or chemistry can achieve under optimal conditions, this information being widely and routinely available to the skilled person. In this regard, it should be understood that not all reads which are produced in a single sequencing run will necessarily result in producing the maximum possible read length. Still further, the comparative length of the millions of forward reads and millions of reverse reads produced in a high throughput bidirectional sequencing step will not be equivalent. Variability between sequence read lengths is usually observed. That is, the forward read lengths may vary from one to the other by up to 5%, as will the reverse read lengths. As detailed hereinbefore, it has been unexpectedly determined that when aligning a series of unpaired forward or unpaired reverse reads which are all derived from the same template molecule, and therefore express the same sequence, currently available alignment software and algorithms will sometimes classify these sequences as being different sequences simply due to the generation of reads with slightly different lengths. In terms of clinical applications where one is screening for minimum residual disease, clonal evolution or the existence or emergence of minor clones, such analysis errors can adversely impact the specificity and/or sensitivity of the result.

As detailed hereinbefore, the target nucleotide sequence is located within the terminal 5′ and/or 3′ contiguous stretch of nucleotides which correspond in length to about 80% of the maximum forward and reverse bidirectional read length. In one embodiment, said maximum read length percentage is 70%-85%, in another embodiment 75%-85% and in yet another embodiment 75%-80%. In still another embodiment said maximum read length percentage is 75%, 76%, 77%, 78%, 79%, 80%, 81%, 82% or 83%. Reference to the target nucleotide sequences being “localised to” the defined contiguous nucleotide region should be understood to mean that the target sequence is located within that region but not necessarily across the entire length of that region. That is, there may be stretches of sequence within the defined region that do not express target sequence. This is more likely to occur where the target nucleotide sequence is small. To the extent that there may be two target nucleotide sequences, these may be distally located at the 5′ and 3′ ends of the template, for example as may occur if a portion of specific V gene segment is located at the 5′ end of the template and some or all of the CDR3 region is located at the 3′ end of the template. It should be understood that if there is only one target nucleotide sequence of interest, then either the 5′ or the 3′ terminal end of the template will not express a target nucleotide sequence. It should also be understood that there may be more than one target nucleotide sequence located within a single defined 5′ or 3′ region. For example, one may screen for both a V gene segment specific sequence and, further, the occurrence of somatic hypermutation within that specific V gene segment sequence. In this case, there are two target nucleotide sequences which are the subject of analysis and these are both located within the defined contiguous nucleotide region at the end of the template DNA.

According to this embodiment, there is provided a method of screening a DNA sample of interest for the expression of one or more target DNA sequences, said method comprising:

(i) spatially isolating on a glass surface a library of individual template DNA molecules derived from said DNA sample, which template DNA molecules have been generated such that the target DNA sequences are localised to the region of contiguous nucleotides at the 5′ and/or 3′ terminal ends of said template, wherein said contiguous nucleotide region corresponds to 75%, 76%, 77%, 78%, 79%, 80%, 81%, 82% or 83% of the maximum forward and reverse read length deliverable by the bidirectional sequencing technology selected for use in step (iii) and wherein the terminal end of said contiguous nucleotide region expresses one or more nucleic acid sequences corresponding to adaptors indexes, barcodes, unique molecular identifiers, sequencing primer hybridisation sites and index sequencing primer hybridisation sites;

(ii) amplifying said spatially isolated template DNA molecules to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read;
and wherein:
(1) said portion is not less than 75%, 76%, 77%, 78%, 79%, 80%, 81%, 82% or 83% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all the nucleic acid sequence results of (a) and the linker sequence is the same for all the nucleic acid sequence results of (b); and

(v) analysing the sequence result.

As detailed hereinbefore, the target nucleotide sequence must be located within a defined 5′ or 3′ terminal contiguous nucleotide region of the template DNA corresponding to about 80% of the maximum theoretical read length of the selected bidirectional sequencing technology. It should be understood that reference to this region of the template is a reference to a defined region, irrespective of whether it is functionally available or not to express the target nucleotide sequence. Accordingly, the contiguous nucleotide region within which the target sequence could actually be located may be less than the equivalent of the maximum read length. For example, to the extent that the template DNA may have been designed to incorporate additional nucleic acid features such as adaptors, indexes, barcodes, primer hybridisation sites and the like (herein referred to as the “adaptor region”), all or some of this stretch of terminal nucleotides is rendered unavailable to the target sequence depending on where the sequencing primer hybridization site is positioned within the adaptor region, since this additional adaptor region necessarily forms part of the bidirectional sequence read. Specifically, the section of adaptor region sequence which is located 3′ to the sequencing primer hybridization site will form part of the sequence read but the section of adaptor sequence which is located 5′ to the primer hybridization site. The skilled person would appreciate that it is conceivable that such non-target nucleic acid features may comprise a contiguous nucleotide length of 10-30 nucleotides, for example, that are located at the terminal 5′ and 3′ positions. To the extent that a bidirectional sequence read is 2×100-150 nucleotides, a region of 10-30 nucleotides which is not available to the target sequence corresponds to a larger proportion of read length which is unusable for maximizing target sequence read length than if the selected sequence read length is 2×200-300 nucleotides. However, as the skilled person would appreciate, the bidirectional read length is not the only consideration in selecting particular instrumentation or chemistry for use. For example, the Illumina MiSeq instrumentation, although offering a bidirectional read length of 2×300 nucleotides, offers a read depth which is more than an order of magnitude less than the NovaSeq instrumentation, which only offers a read length of 2×150. Where one is seeking to apply this method to MRD analysis, for instance, sequence depth becomes a crucial factor. Accordingly, the ability to now select any high throughput bidirectional sequencing instrumentation and chemistry for use, irrespective of whether overlapping bidirectional reads can be generated, has significantly widened the scope of application of this class of technology.

In one embodiment, there is provided a method of screening a DNA sample of interest for the expression of one or more target DNA sequences, said method comprising:

(i) spatially isolating on a glass surface a library of individual template DNA molecules derived from said DNA sample, which template DNA molecules have been generated such that the target DNA sequences are localised to the 120 contiguous nucleotides at the 5′ and/or 3′ terminal ends of said template but wherein the 20 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites;

(ii) amplifying said spatially isolated template DNA molecules to generate clusters of amplicons wherein each cluster is generated from an individual spatially isolated template DNA molecule;

(iii) bidirectionally sequencing one or more amplicons of one or more clusters wherein using sequencing chemistry which produces a maximum forward read length of 150 nucleotides and a maximum reverse length of 150 nucleotides;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read; and wherein said portion is 120 nucleotides of each of the forward and reverse read length and the linker sequence is the same for all the nucleic acid sequence results of
(a) and the linker sequence is the same for all the nucleic acid sequence results of (b); and

(v) analysing the sequence result.

In another embodiment, said target DNA sequences are localised to the 125 contiguous nucleotides at the 5′ and/or 3′ terminal ends of said template but wherein up to the 30 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites.

It would be appreciated that it is well within the skill of the person in the art to generate a DNA template where the one or more target nucleotide sequences are localised to the 5′ and/or 3′ ends of the template as hereinbefore defined. Since the overall length of the DNA template is now largely inconsequential, the skilled person need only identify the target sequences and then determine how to incorporate them into a DNA template at the correct position. Where there is only one target sequence of interest, it may be possible to generate a template by simply cleaving the DNA of the biological sample close to the target sequence, for example using an appropriate restriction enzyme, and then either ligating any necessary adaptor region to the fragment or amplifying the fragments using consensus primers which comprise the adaptor region sequence at the terminal end of the primer as a non-hybridizing tail region and thereby incorporate the adaptor region into the amplification product to generate the template library. Alternatively, one may perform amplification of the DNA sample using primers wherein either the forward or reverse primer flanks the target sequence and thereby enables its amplification while the other primer binds to any suitable region of the DNA to enable PCR to proceed. These primers may incorporate the adaptor region sequence at the terminal end of the primer as a non-hybridizing region, and thereby incorporate the adaptor region into the amplification product in a single step, or a second round amplification may be performed which uses consensus primers directed to the first round amplification product to introduce the adaptor region. Where more than one target sequence is sought to be analysed, the skilled person can design amplification primers which flank the 5′ end of the upstream target nucleotide sequence and the 3′ end of the downstream target nucleotide sequence. The length of the intervening sequence is not relevant provided that the target nucleotides sequences which are selected for analysis can be localised to the terminal 5′ and 3′ regions as hereinbefore defined. Designing primers which will flank and amplify one or more target nucleotide sequences is a routine and simple procedure. The skilled person will appreciate that by positioning an amplification primer such that it flanks the target sequence as closely as possible to where the target nucleotide sequence either commences or ends, depending on the position of the target sequences relative to one another and the orientation of the primer in issue, one can maximise the length of target nucleotide sequence which can be localised to the defined 5′ and/or 3′ ends of the DNA template and which can thereby be sequenced. In this regard, one may design the primer such that it hybridizes within the target sequence itself, and therefore forms part of the amplified target sequence nucleotide sequence, in which case the length of the primer sequence will form part of the 5′ and/or 3′ DNA template region which is sequenced. Where the primer hybridises outside the target region, one may elect to design the primer sequence with a cleavage site at its 3′ end which enables the primer sequence to be cleaved off the amplicon in a site directed fashion. In any of these examples, the adaptor region may be introduced in either a single or two step procedure as described above. In yet another example, one may seek to generate the template DNA using non-PCR based methods, such as splicing a region of DNA expressing the target nucleotide sequence into a vector and amplifying the vector via host cell replication. DNA templates generated in this way would require excision from the vector prior to facilitating their attachment to a solid support.

As detailed hereinbefore, the method of the present invention is directed to a means of applying high throughput bidirectional sequencing to screening a nucleic acid sample even where overlapping bidirectional reads are not obtainable due to the template DNA being longer than the combined read length of the sequencing chemistry. This is achieved, in part, by spatially isolating the individual template DNA molecules on a solid support such that amplification can be performed by any suitable method to generate clusters of amplicons. Reference to an “amplicon” in this regard is a reference to the amplified copies of the template DNA and/or its complementary sequence. Reference to a “cluster” is therefore intended as a reference to the colony of amplicons which are generated and anchored proximally to the template DNA such that a colony of clonal target sequences and clonal complementary sequences is generated around a single template DNA. Methods for performing cluster DNA are well known to the skilled person and can be performed as a matter of routine procedure. An exemplary method of achieving such cluster amplification is bridge amplification. In this method, once the template DNA, comprising adaptor sequences at both the 5′ and 3′ ends, has been immobilised on the solid support at the appropriate density, nucleic acid clusters can be generated by carrying out an appropriate number of cycles of amplification on the immobilised template DNA such that each colony comprises multiple copies of the original immobilised template DNA and its complementary sequence. One cycle of amplification consists of the steps of hybridisation, extension and denaturation and these steps are generally performed using reagents and conditions well known in the art for PCR. A typical amplification reaction comprises subjecting the solid support and attached template DNA to conditions which induce primer hybridisation and extension in the presence of a nucleic acid polymerase together with a supply of nucleoside triphosphate molecules or any other nucleotide precursors, for example modified nucleoside triphosphate molecules. The primer will be extended by the addition of nucleotides complementary to the template DNA. Examples of nucleic acid polymerases which can be used in the present invention are DNA polymerase (Klenow fragment, T4 DNA polymerase), heat-stable DNA polymerases from a variety of thermostable bacteria (such as Taq, VENT, Pfu, Tfl DNA polymerases) as well as their genetically modified derivatives (TaqGold, VENTexo, Pfu exo). A combination of RNA polymerase and reverse transcriptase can also be used to generate the amplification of a DNA colony. Preferably the nucleoside triphosphate molecules used are deoxyribonucleotide triphosphates, for example dATP, dTTP, dCTP, dGTP. The nucleoside triphosphate molecules may be naturally or non-naturally occurring.

Subsequently to the hybridisation and extension steps, two immobilised nucleic acids will be present, the first being the template strand and the second being a nucleic acid strand complementary thereto. Both of these nucleic acid molecules are then able to initiate further rounds of amplification via the formation of a bridge and hybridisation of the non-immobilized end of the amplicon with its complementary immobilized anchor. Such further rounds of amplification will result in a nucleic acid cluster comprising multiple immobilised clonal copies of the template strand and its complementary sequence. The initial immobilisation of the template DNA means that the template DNA can only form a bridge and hybridise to adaptor anchors located at a distance within the length of the template DNA. Thus the boundary of the cluster is limited to a relatively local area in which the initial template DNA was immobilised. Clearly, once more copies of the template strand and its complement have been synthesised by carrying out further rounds of amplification, the cluster being generated will be able to be extended further, although the boundary of the cluster formed is still limited to a relatively local area in which the initial template DNA was immobilised. The subject amplification may be performed qualitatively or quantitatively.

In one embodiment, said amplification is bridge amplification.

According to this embodiment, there is provided a method of screening a DNA sample of interest for the expression of one or more target DNA sequences, said method comprising:

(i) spatially isolating on a glass surface a library of individual template DNA molecules derived from said DNA sample, which template DNA molecules have been generated such that the target DNA sequences are localised to the region of contiguous nucleotides at the 5′ and/or 3′ terminal ends of said template, wherein the terminal end of said contiguous nucleotide region expresses one or more nucleic acid sequences corresponding to indexes, barcodes, unique molecular identifiers, sequencing primer hybridisation sites and index sequencing primer hybridisation sites;

(ii) amplifying said spatially isolated template DNA molecules by bridge amplification to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read;
and wherein:
(1) said portion is not less than 75% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all the nucleic acid sequence results of (a) and the linker sequence is the same for all the nucleic acid sequence results of (b); and

(v) analysing the sequence result.

Preferably said glass surface is a glass slide or a flow cell.

In another embodiment said nucleic sample of interest comprises B and/or T cell DNA and said one or more target nucleotide sequences are one or more rearranged V, D or J gene segments.

In yet another embodiment said target nucleotide sequences are the DJ or VDJ rearrangements of IgH, TCR β or TCR δ or the VJ rearrangement of Igκ, Igλ, TCRα or TCRγ. In still another embodiment, said rearrangement is a kappa deleting element rearrangement.

In still yet another embodiment, said target nucleotide sequences are a V gene segment region, such as a region predisposed to undergoing hypermutation and/or a J gene segment region encoding a portion of the CDR3.

In yet still another embodiment, said target nucleotide sequences are the gene segment regions encoding all or some of the V leader sequence, the V region predisposed to somatic hypermutation, IgH FR1, IgH FR2 or IgH FR3.

In another embodiment, said contiguous nucleotide region of step (i) corresponds to about 80% of the maximum forward and reverse read length deliverable by the bidirectional sequencing technology selected for use in step (iii).

In a further embodiment said contiguous nucleotide region corresponds to 75%, 76%, 77%, 78%, 79%, 80%, 81%, 82% or 83% of the maximum forward and reverse read length deliverable by the bidirectional sequencing technology selected for use in step (iii) and said forward and reverse read portions is not less than 75%, 76%, 77%, 78%, 79%, 80%, 81%, 82% or 83% of the maximum forward and reverse read length deliverable by the bidirectional sequencing technology selected for use in step (iii).

In yet another embodiment, said target DNA sequences are localised to the 120 contiguous nucleotides at the 5′ and/or 3′ terminal ends of said template but wherein the 20 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites.

In yet still another embodiment, said target DNA sequences are localised to the 125 contiguous nucleotides at the 5′ and/or 3′ terminal ends of said template but wherein up to the 30 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites.

Subsequently to cluster formation, bidirectional sequencing of one or more amplicons of one or more clusters is performed. It is anticipated, however, that in most situations there will be effected parallel bidirectional sequencing of all clusters and all amplicons within those clusters. Any high-throughput method for the bidirectional sequencing of nucleic acids can be used in the method of the invention. In one example, sequencing by synthesis using reversibly terminated labelled nucleotides is applied. As detailed hereinbefore, and without limiting the present invention to any one theory or mode of action, in one embodiment of bidirectional sequencing which uses reversibly terminated labelled nucleotides, subsequently to clonal amplification the reverse strands are washed off the solid support, leaving only forward (template) strands. Sequencing then commences. Primers attach to the forward strands and a polymerase adds fluorescently tagged nucleotides to the DNA strand. Only one base is added per round. A reversible terminator which is present on every nucleotide prevents multiple additions in one round. Each of the four bases produces a unique emission, and after each round, the instrumentation which is used records which base was added based on the emitted fluorescence. Once the forward DNA strand has been read and the sequence read washed away, the reverse strand is generated via another round of bridge amplification. The forward strand is then washed away and the process of sequence by synthesis repeats for the reverse strand. In this way, bidirectional sequencing is achieved.

In one embodiment, said method is sequencing by synthesis using reversibly terminated labelled nucleotides.

According to this embodiment, there is provided a method of screening a DNA sample of interest for the expression of one or more target DNA sequences, said method comprising:

(i) spatially isolating on a glass surface a library of individual template DNA molecules derived from said DNA sample, which template DNA molecules have been generated such that the target DNA sequences are localised to the region of contiguous nucleotides at the 5′ and/or 3′ terminal ends of said template, wherein the terminal end of said contiguous nucleotide region expresses one or more nucleic acid sequences corresponding to indexes, barcodes, unique molecular identifiers, sequencing primer hybridisation sites and index sequencing primer hybridisation sites;

(ii) amplifying said spatially isolated template DNA molecules by bridge amplification to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon and wherein said bidirectional sequencing is sequencing by synthesis using reversibly terminated labelled nucleotides;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read;
and wherein:
(1) said portion is not less than 75% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all the nucleic acid sequence results of (a) and the linker sequence is the same for all the nucleic acid sequence results of (b); and

(v) analysing the sequence result.

Preferably said glass surface is a glass slide or a flow cell.

In another embodiment said nucleic sample of interest comprises B and/or T cell DNA and said one or more target nucleotide sequences are one or more rearranged V, D or J gene segments.

In yet another embodiment said target nucleotide sequences are the DJ or VDJ rearrangements of IgH, TCR β or TCR δ or the VJ rearrangement of Igκ, Igλ, TCRα or TCRγ. In still another embodiment said rearrangement is a kappa deleting element rearrangement.

In still yet another embodiment, said target nucleotide sequences are a V gene segment region, such as a region predisposed to undergoing hypermutation and/or a J gene segment region encoding a portion of the CDR3.

In yet still another embodiment, said target nucleotide sequences are the gene segment regions encoding all or some of the V leader sequence, the V region predisposed to somatic hypermutation, IgH FR1. IgH FR2 or IgH FR3.

In another embodiment, said contiguous nucleotide region of step (i) corresponds to about 80% of the maximum forward and reverse read length deliverable by the bidirectional sequencing technology selected for use in step (iii).

In a further embodiment said contiguous nucleotide region corresponds to 75%, 76%, 77%, 78%, 79%, 80%, 81%, 82% or 83% of the maximum forward and reverse read length deliverable by the bidirectional sequencing technology selected for use in step (iii) and said forward and reverse read portions is not less than 75%, 76%, 77%, 78%, 79%, 80%, 81%, 82% or 83% of the maximum forward and reverse read length deliverable by the bidirectional sequencing technology selected for use in step (iii).

In yet another embodiment, said target DNA sequences are localised to the 120 contiguous nucleotides at the 5′ and/or 3′ terminal ends of said template but wherein the 20 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites.

In yet still another embodiment, said target DNA sequences are localised to the 125 contiguous nucleotides at the 5′ and/or 3′ terminal ends of said template but wherein up to the 30 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites.

As detailed hereinbefore, the method of the present invention is predicated on the development of a means of analysing non-overlapping bidirectional sequence reads which provides accurate and reproducible results. This development is based, in part, on the unexpected determination that although one or more clusters of forward or reverse reads have derived from the same template sequence, and therefore express the same sequence read results, any difference in the lengths of the reads alone will result in current analytical software categorising these reads as being different, despite the fact that most of the sequence of the read will be identical between these reads. The added complication that sequencing errors become more frequent toward the 3′ end of a sequencing read introduces further complexity into analysing the result. Where the bidirectional sequence reads comprise overlapping and complementary 3′ ends, the issue of an individual read length is rendered moot since the reads are taped together prior to alignment and further analysis. Even the issue of sequencing errors is mitigated since the information from the strand which is complementary to the strand expressing the sequencing anomaly assists in determining whether any such sequence differences are real or not. This is not possible when analysing a read for which an overlapping complementary strand read is not available. It is for this reason that current teaching in relation to high throughput bidirectional sequencing is that the template DNA should always be designed such that its length is compatible with the read length of the instrumentation which is proposed to be used. Still further, as the skilled person would know, although bidirectional sequencing instrumentation provides a theoretical maximum sequence read length, the actual reads which are obtained will not necessarily precisely reflect that read length and the actual read length which is obtained may vary by as much as 5% or so between reads.

In accordance with the present method, the forward and reverse reads are identified for one or more of the clusters which have been sequenced. By “identified” is meant that the sequence information for the forward and reverse reads which are co-localised to a single cluster are determined. In this regard, where multiplexed high throughput screening has been performed, the skilled person may elect to initially identify the forward and reverse read sequence information for some clusters but not for all. For example, one many elect to demultiplex the results, if a multiplexed reaction has been performed in order to analyse multiple patient samples, and one may initially analyse the information for one patient and not the others. This demultiplexing step is effected via the use of patient specific indexes or barcodes. Alternatively, if more than one target sequence was screened for using distinct pairs of primers, which may themselves have been designed to be distinguishable via an index or other suitable means which would be well known to the person in the art, one may elect to initially analyse just one of these target nucleotide sequences. In one embodiment, all clusters for which bidirectional sequencing information has been produced are analysed. In this regard, the analysis of the sequence reads and the generation and analysis of a sequence result, as hereinafter described in more detail, can be performed in any convenient manner. For example, one may manually review the sequence data or one may use a suitable algorithm to effectively automate one or more of the analysis steps described in step (iv). Alternatively, one may use a combination of methods and algorithms to perform the steps described in step (iv). It should be understood that this analysis, including the generation of the sequence result, will most conveniently be performed in silico.

As detailed hereinbefore, the forward and reverse reads for an individual template DNA molecule which has undergone cluster amplification and bidirectional sequencing in accordance with the present method are identifiable based on the co-localisation of these reads to the position of a single cluster on the solid support. However, these reads will not exhibit an overlapping and complementary sequence region at their 3′ ends. Once these “paired” reads have been identified, the nucleic acid sequence result can be generated. By “sequence result” is meant the sequence which is assembled from the forward and reverse reads and which is then in a form suitable for the final analysis step, such as alignment of the sequence results of each of the clusters to assess the clonality or diversity of the DNA sample of interest, alignment of the sequence results to a reference sequence to further classify the sequence (e.g. to determine the specific identity of a V, D or J gene segment if the template DNA was amplified using gene family or consensus primers), identifying the occurrence and nature of a hypermutation, indel, DNA breakpoint, SNP or the like, assessing clonal evolution or determining the emergence of a new clone. In another example, one may seek to identify a patient specific sequence in the context of MRD monitoring since this may indicate the re-emergence of disease. It should be understood that the sequence result may include a portion of the 5′ and 3′ adaptor region, depending on where the sequencing primer hybridisation site was positioned. In this regard, the skilled person may elect to cleave off this additional sequence such that the sequence result includes only the sequence corresponding to the DNA sample of interest, together with the intervening linker region. However, the skilled person may also determine that this is unnecessary and the sequence result will retain this additional sequence at its 5′ and 3′ ends since it is identifiable.

Said nucleic acid sequence result is generated by assembling, usually in silico, a portion of the 5′ contiguous nucleic acid sequence of the forward read and the reverse read, which may or may not include any terminal nucleotides which correspond to the adaptor region. Reference to “portion” should be understood as a reference to some, but not necessarily all, of the forward and reverse read sequence length, although in relation to shorter reads, one may use the entire sequence. The subject portion which is to be utilised will be determined by the skilled person but it will not be less than about 80% of the maximum read deliverable by the selected bidirectional sequencing technology and the portion selected will be the same for all forward reads and all reverse reads which are analysed for a given DNA sample of interest. Reference to “the maximum forward and reverse read length deliverable by the selected bidirectional sequencing technology” should be understood to have the same meaning as detailed earlier. By selecting a portion within these parameters, it has been determined that this provides sufficient target nucleotide sequence data to achieve specificity in terms of the target sequence information of interest and sequence accuracy in terms of removal of sufficient of the 3′ sequence data which exhibits an increased probability of containing sequence errors, thereby enabling both sensitive and specific screening outcomes for the DNA sample of interest. In terms of determining the portion which will be used for the screening of a DNA sample, this will be well within the skill of the person in the art to determine when considered in light of the teaching provided herein. To the extent that a multiplexed assay is performed with samples from multiple patients, multiple different tissues and/or is directed to different target sequences, for example, the skilled person may determine a different portion length as between categories of results. However, in the context of a single DNA sample source, the portion will be the same for all forward sequence reads and the same for all reverse sequence reads. In this regard, the portion length which is selected for use with the forward reads need not be the same as the portion length which is selected for the reverse reads. By ensuring that the nucleic acid length of the forward and reverse portions are the same as between all the forward read portions and all the reverse read portions, the unexpected incidence of potential misclassification of clonal sequences as being different sequences due only to the fact that one sequence is longer than the other is obviated.

Said forward and reverse read portions are assembled to generate the sequence read result by linking the 3′ end of the forward read to the reverse read-derived sequence information via a nucleic acid linker. In this regard, the skilled person would appreciate that the sequences of the forward and reverse reads correspond to the sequences of the 5′ end of the template/forward strand the 5′ end of the complementary/reverse strand, respectively. Accordingly, if these reads were to extend along the full length of the sequence to which they were hybridised, the two reads would be complementary. Accordingly, in the context of the present invention, which is directed to taping the 5′ and 3′ ends of the template DNA and the 5′ and 3′ ends of the strand which is complementary to the template strand, it is necessary to determine the complementary sequence to each of the forward and reverse read sequences, which can be achieved easily and quickly in silico, and to tape the forward read sequence to the complement of the reverse read sequence. Similarly, the complement of the forward read sequence is taped to the reverse read sequence. This will then generate a template sequence result, albeit only the 5′ and 3′ end sequences, and a corresponding sequence result for the strand which is complementary to the template strand.

Reference to a “nucleic acid linker” should be understood as a reference to a nucleic acid sequence, preferably a linear sequence, which is attached to the 3′ ends of the forward and reverse read portions and to the 5′ ends of the sequences which are complementary to the forward and reverse read portions so as to form a single linear contiguous nucleic acid sequence where the 3′ end of the forward read sequence is linked to the sequence complementary to the reverse read sequence and the 3′ end of the reverse read sequence is linked to the the complement to the forward read sequence. The nucleotides of the linker may be any naturally or non-naturally occurring nucleotide, although to the extent that this aspect of the invention is performed in silico, the actual chemical structure of the nucleotides of the assembled sequence result is less important than that the in silico functional information in relation to these nucleotides is such that they are interpreted and analysed as if they function in their corresponding physical form, such as exhibiting correct complementary base pairing if that was relevant. Reference to “naturally and non-naturally” occurring nucleotides should have the same meaning as hereinbefore provided. In one embodiment said nucleic acid linker is Nₓ, where N represent a natural or non-natural nucleotide and x represents the number of contiguous nucleotides in the linker. In terms of the nature of the linker sequence itself, this may be a random sequence, although if a randomly generated sequence is used, it must be the same for all sequence results since differences in the linker sequence used for the forward and reverse read pairs which are assembled, and which are otherwise clonally derived and therefore identical, would result in these sequences being classified as being different due to the linker sequence variation. It would also mean that comparisons between the sequence results of a single DNA sample, such as in the context of immune receptor diversity, would be meaningless. Preferably, where the subject sequences are concatenated in silico, said N nucleotide is simply designated N and is thereby distinct and discernible relative to the naturally occurring nucleotides of A, T, G and C. The length of the linker sequence may be any suitable length which is determined by the skilled person. In this regard, it has been determined that number of nucleotides in the linker should not be too few, since a nucleotide “linker” of only 1 or 2 Ns may be interpreted as a random nucleotide insert, and thereby misalign the sequence, rather than being interpreted as the linker. In one embodiment, said linker is 5-30 nucleotides in length, preferably 5-25 and more preferably 5-20. In another embodiment, the length of said linker is 5, 6, 7, 8, 9, 10, 11, 12, 13, 14, 15 or 16 nucleotides.

In accordance with this embodiment, there is provided a method of screening a DNA sample of interest for the expression of one or more target DNA sequences, said method comprising:

(i) spatially isolating on a glass surface a library of individual template DNA molecules derived from said DNA sample, which template DNA molecules have been generated such that the target DNA sequences are localised to the region of contiguous nucleotides at the 5′ and/or 3′ terminal ends of said template, wherein the terminal end of said contiguous nucleotide region expresses one or more nucleic acid sequences corresponding to indexes, barcodes, unique molecular identifiers, sequencing primer hybridisation sites and index sequencing primer hybridisation sites;

(ii) amplifying said spatially isolated template DNA molecules by bridge amplification to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon and wherein said bidirectional sequencing is sequencing by synthesis using reversibly terminated labelled nucleotides;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read;
and wherein:
(1) said portion is not less than 75% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is 5-30 nucleotides in length and is the same for all the nucleic acid sequence results of (a) and the linker sequence is the same for all the nucleic acid sequence results of (b); and

(v) analysing the sequence result.

Preferably said glass surface is a glass slide or a flow cell.

In another embodiment said nucleic sample of interest comprises B and/or T cell DNA and said one or more target nucleotide sequences are one or more rearranged V, D or J gene segments.

In yet another embodiment said target nucleotide sequences are the DJ or VDJ rearrangements of IgH, TCR β or TCR δ or the VJ rearrangement of Igκ, Igλ, TCRα or TCRγ. In still another embodiment, said rearrangement is a kappa deleting element rearrangement.

In still yet another embodiment, said target nucleotide sequences are a V gene segment region, such as a region predisposed to undergoing hypermutation and/or a J gene segment region encoding a portion of the CDR3.

In yet still another embodiment, said target nucleotide sequences are the gene segment regions encoding all or some of the V leader sequence, the V region predisposed to somatic hypermutation, IgH FR1, IgH FR2 or IgH FR3.

In another embodiment, said contiguous nucleotide region of step (i) corresponds to about 80% of the maximum forward and reverse read length deliverable by the bidirectional sequencing technology selected for use in step (iii).

In a further embodiment said contiguous nucleotide region corresponds to 75%, 76%, 77%, 78%, 79%, 80%, 81%, 82% or 83% of the maximum forward and reverse read length deliverable by the bidirectional sequencing technology selected for use in step (iii) and said forward and reverse read portions is not less than 75%, 76%, 77%, 78%, 79%, 80%, 81%, 82% or 83% of the maximum forward and reverse read length deliverable by the bidirectional sequencing technology selected for use in step (iii).

In yet another embodiment, said target DNA sequences are localised to the 120 contiguous nucleotides at the 5′ and/or 3′ terminal ends of said template but wherein the 20 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites.

In yet still another embodiment, said target DNA sequences are localised to the 125 contiguous nucleotides at the 5′ and/or 3′ terminal ends of said template but wherein up to the 30 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites.

In another embodiment, said linker is 5-25 nucleotides in length. In still another embodiment said linker is 5-20 nucleotides in length. In a further embodiment, the length of said linker is 5, 6, 7, 8, 9, 10, 11, 12, 13, 14, 15 or 16 nucleotides, most preferably 9, 10, 11 or 12 nucleotides in length.

Once the sequence result is assembled, the assembled sequences can be analysed. The type of analysis which is performed will be decided by the skilled person and will depend on the nature of the information which is sought. For example, one may mine these results to identify the presence, or not, of a specific mutation or other sequence feature, such as a specific V(D)J immunoglobulin or TCR rearrangement. This may be useful for diagnostic or MRD purposes or to determine the relative effectiveness of treatment. Some diseases are identified by the presence of a specific mutation (e.g. Flt3 or NPM1), hypermutation, indel, gene breakpoint (e.g. BCR-ABL) or the like. Alternatively, rather than screening for the presence of a prior known target sequence, one may be seeking to survey the diversity of sequences of a gene region of interest, which sequence information can then be used to track the progress and/or evolution of a disease. For example, white blood cell neoplasias, which arise from the neoplastic transformation of a single white blood cell, lend themselves to identification and tracking based on identifying the unique V, D and/or J rearrangement of the neoplastic cell. This can be particularly useful for assessing minimum residual disease. Due to the huge diversity of the immune cell repertoire, virtually every white blood cell exhibits a unique immunoglobulin or TCR rearrangement. By identifying one or more of the specific gene segments which have rearranged in a neoplastic population, a specific cell can be tracked. In terms of the application of the present invention, one may also screen the DNA of a biological sample to assess the diversity of a specific rearrangement, such as an IgH VJ rearrangement. If all of the rearranged IgH VJ sequences from a blood or bone marrow sample are screened, the alignment of the sequence results will provide either a qualitative or quantitative readout of the diversity of the IgH VJ gene segment rearrangements. This can be very useful in the context of surveying the immune system to determine the status or progression of immunotherapy, infection, transplantation, autoimmunity, allergy, immunodeficiency or any other situation where there might be value in assessing whether T or B cell clonal expansion has occurred as an indicator of immune activity (either desirable or not). If a clone is present, indicating the expansion of a clonal population (for example due to the acute immune response to a pathogen or autoantigen), an increase in the number of sequence reads corresponding to a single specific rearrangement, relative to the otherwise heterogeneous background array of rearrangement at the IgH VJ locus, will be evident. The identification of the existence of this clone allows the specific gene segment rearrangement to be identified and for that clone to be tracked. This can be particularly important in the context of autoimmunity. If multiple clones are expanding, this can indicate a wide ranging immune response, such as a response to multiple antigens in the context of infection, transplantation or allergy.

In terms of the sequence analysis performed herein, the multiple identical sequence results for a single cluster are aligned and identical sequences are merged into a single sequence result. Non-identical sequences within a cluster are discarded on the basis that if they are different to the sequence of other amplicons from the same cluster, then they likely contain a sequencing error. Complementary sequences may be paired in order to generate DNA duplex results. The single or double stranded sequences between the clusters are then aligned. In one example, tolerance of 2 or 3 nucleotide differences between sequences of different clusters is a threshold under which those sequence may be classified as being derived from a clonal population which is present in the starting DNA sample of interest. The relative or actual proportions (depending on whether the amplification was performed quantitatively or not) are then assessed, for example to determine whether there exists evidence of the expansion of a clone or whether a specific sequence (such as one relevant for MRD assessment) is present.

According to this embodiment, said analysis comprises aligning the nucleic acid sequence results generated in step (iv) and determining the expression of the target nucleic acid sequences of interest.

The present method can therefore be used in diagnosis, prognosis, classification, prediction of disease risk, detection of recurrence of disease, immune surveillance or monitoring of prophylactic or therapeutic efficacy in the context or any disease or non-disease state which can be characterised by the expression of one or more target nucleotide sequences. Still further, this method has application in any other context where the analysis of sequences in certain target DNA and RNA regions or screening for the presence of specific target DNA and RNA sequences is necessitated, such as in the context of research and development. For example, the present invention provides a solution to current and emerging needs that scientists and the biotechnology industry are seeking to address in the fields of genomics, pharmacogenomics, drug discovery, food characterization and genotyping.

Using lymphoid neoplasia as a non-limited example, the present invention provides methods for determining whether a mammal (e.g. a human) has neoplasia, whether a biological sample taken from a mammal contains neoplastic cells or DNA derived from neoplastic cells, estimating the risk or likelihood of a mammal developing a neoplasm, monitoring the efficacy of anti-cancer treatment or selecting the appropriate treatment in a mammal with cancer. Such methods are based on the determination that lymphoid neoplasias are characterised by the clonal expansion of a cell expressing a unique V(D)J rearrangement.

The method of the invention can be used to evaluate individuals known or suspected to have neoplasia, or as a routine clinical test in an individual not necessarily suspected to have a neoplasia. Further, the present methods may be used to assess the efficacy of a course of treatment. For example, the efficacy of an anti-cancer treatment can be assessed by monitoring DNA methylation over time in a mammal having a lymphoid cancer. For example, a reduction or absence of a clonal population characterised by a specific target nucleotide sequence in a biological sample taken from a mammal following treatment indicates efficacious treatment.

The method of the present invention is therefore useful as a one-time test or as an on-going monitor of an individual, whether in the context of a lymphoid neoplasia or any other application as hereinbefore described. In these situations, screening for a target sequence is a valuable indicator of the status of an individual, for example the status of their immune system.

Accordingly, in another aspect there is provided a method of diagnosing, monitoring or otherwise screening for a condition in a patient, which condition is characterised by the expression of one or more target nucleotide sequences, said method comprising:

(i) spatially isolating on a solid support a library of individual template DNA molecules derived from a nucleic acid sample, which template DNA molecules have been generated such that the target nucleotide sequences are localised to the region of contiguous nucleotides at the 5′ and/or 3′ terminal ends of said template;

(ii) amplifying said spatially isolated template DNA molecules to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read;
and wherein:
(1) said portion is not less than 75% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all the nucleic acid sequence results of (a) and the linker sequence is the same for all the nucleic acid sequence results of (b); and

(v) analysing the sequence result.

Reference to a “nucleic acid sample” should be understood as a reference to any sample of DNA derived from any organism, such as a plant, animal or microorganism or any recombinant, synthetic or artificial source such as, but not limited to, cellular material, blood, mucus, faeces, urine, tissue biopsy specimens or fluid which has been introduced into the body of an animal and subsequently removed (such as, for example, the saline solution extracted from the lung following lung lavage or the solution retrieved from an enema wash), microorganism (eg. bacteria, viruses, parasites), tissue culture or recombinant DNA processes. The biological sample which is tested according to the method of the present invention may be tested directly or may require some form of treatment prior to testing. For example, a biopsy sample may require homogenisation prior to testing. Further, to the extent that the biological sample is not in liquid form it may require the addition of a reagent, such as a buffer, to mobilise the sample.

To the extent that the target DNA is present in a sample, the sample may be directly tested or else all or some of the nucleic acid material present in the sample may be isolated prior to testing. It is within the scope of the present invention for the target nucleic acid molecule to be pre-treated prior to testing, for example inactivation of live virus or being run on a gel. It should also be understood that the sample may be freshly harvested or it may have been stored (for example by freezing) prior to testing or otherwise treated prior to testing (such as by undergoing culturing). The sample may also have undergone in vitro culture or manipulation (such as immortalisation or recombination) to generate a cell line or cell culture.

The choice of what type of sample is most suitable for testing in accordance with the method disclosed herein will be dependent on the nature of the situation, such as the nature of the condition being monitored. For example, in a preferred embodiment a neoplastic condition is the subject of analysis. If the neoplastic condition is a lymphoid leukaemia, a blood sample, lymph fluid sample or bone marrow aspirate would likely provide a suitable testing sample. Where the neoplastic condition is a lymphoma, a lymph node biopsy or a blood or marrow sample would likely provide a suitable source of tissue for testing. Consideration would also be required as to whether one is monitoring the original source of the neoplastic cells or whether the presence of metastases or other forms of spreading of the neoplasia from the point of origin is to be monitored. In this regard, it may be desirable to harvest and test a number of different samples from any one mammal. In another example, in the case of infection one may test for either or both of cell expansion and microorganism clonal proliferation, such as viral expansion. Choosing an appropriate sample for any given detection scenario would fall within the skills of the person of ordinary skill in the art.

The term “mammal” to the extent that it is used herein includes humans, primates, livestock animals (e.g. horses, cattle, sheep, pigs, donkeys), laboratory test animals (e.g. mice, rats, rabbits, guinea pigs), companion animals (e.g. dogs, cats) and captive wild animals (e.g. kangaroos, deer, foxes). preferably, the mammal is a human or a laboratory test animal. Even more preferably the mammal is a human.

The nucleic acid sample which is tested may be cell free DNA, such as is found in the circulation in the context of some disease conditions, or it may be derived from a cell.

Reference to “cell or cells” should be understood as a reference to all forms of cells from any species and to mutants or variants thereof. In one embodiment, the cell is a lymphoid cell, although the method of the present invention can be performed on any type of cell which may have undergone a partial or full immunoglobulin or TCR rearrangement. Without limiting the present invention to any one theory or mode of action, a cell may constitute an organism (in the case of unicellular organisms) or it may be a subunit of a multicellular organism in which individual cells may be more or less specialised (differentiated) for particular functions. All living organisms are composed of one or more cells. The subject cell may form part of the biological sample which is the subject of testing in a syngeneic, allogeneic or xenogeneic context. A syngeneic context means that the clonal cell population and the biological sample within which that clonal population exists share the same MHC genotype. This will most likely be the case where one is screening for the existence of a neoplasia in an individual, for example. An “allogeneic” context is where the subject clonal population in fact expresses a different MHC to that of the individual from which the biological sample is harvested. This may occur, for example, where one is screening for the proliferation of a transplanted donor cell population (such as an immunocompetent bone marrow transplant) in the context of a condition such as graft versus host disease. A “xenogeneic” context is where the subject clonal cells are of an entirely different species to that of the subject from which the biological sample is derived. This may occur, for example, where a potentially neoplastic donor population is derived from xenogeneic transplant.

“Variants” of the subject cells include, but are not limited to, cells exhibiting some but not all of the morphological or phenotypic features or functional activities of the cell of which it is a variant. “Mutants” includes, but is not limited to, cells which have been naturally or non-naturally modified such as cells which are genetically modified.

In one embodiment, said condition is characterised by a clonal population of cells or microorganisms.

By “clonal” is meant that the subject population of cells or microorganisms has derived from a common cellular origin. For example, a population of neoplastic cells is derived from a single cell which has undergone transformation at a particular stage of differentiation. In this regard, a neoplastic cell which undergoes further genomic rearrangement or mutation to produce a genetically distinct population of neoplastic cells is also a “clonal” population of cells, albeit a distinct clonal population of cells. In another example, a T or B lymphocyte which expands in response to an acute or chronic infection or immune stimulation is also a “clonal” population of cells within the definition provided herewith. In yet another example, the clonal population of cells is a clonal microorganism population or a viral clone, such as a drug resistant clone which has arisen within a larger microorganismal population. Preferably, the subject clonal population of cells is a neoplastic population of cells or a clonal immune cell population.

In one embodiment, said clonal cells are a population of clonal lymphoid cells.

It should be understood that reference to “lymphoid cell” is a reference to any cell which has rearranged at least one germ line set of immunoglobulin or TCR variable region gene segments. The immunoglobulin variable region encoding genomic DNA which may be rearranged includes the variable regions associated with the heavy chain or the κ or λ light chain while the TCR chain variable region encoding genomic DNA which may be rearranged include the α, β, γ and δ chains. In this regard, a cell should be understood to fall within the scope of the “lymphoid cell” definition provided the cell has rearranged the variable region encoding DNA of at least one immunoglobulin or TCR gene segment region. It is not necessary that the cell is also transcribing and translating the rearranged DNA. In this regard, “lymphoid cell” includes within its scope, but is in no way limited to, immature T and B cells which have rearranged the TCR or immunoglobulin variable region gene segments but which are not yet expressing the rearranged chain (such as TCR-thymocytes) or which have not yet rearranged both chains of their TCR or immunoglobulin variable region gene segments. This definition further extends to lymphoid-like cells which have undergone at least some TCR or immunoglobulin variable region rearrangement but which cell may not otherwise exhibit all the phenotypic or functional characteristics traditionally associated with a mature T cell or B cell. Accordingly, the method of the present invention can be used to monitor neoplasias of cells including, but not limited to, lymphoid cells at any differentiative stage of development, activated lymphoid cells or non-lymphoid/lymphoid-like cells provided that rearrangement of at least part of one variable region gene region has occurred. It can also be used to monitor the clonal expansion which occurs in response to a specific antigen.

In another embodiment, said condition is characterised by one or more target nucleotide sequences which are expressed by an immune cell. In another embodiment said condition is characterised by the expression of one or more rearranged V, D or J gene segment sequence characteristics.

In accordance with this embodiment there is provided a method of diagnosing, monitoring or otherwise screening for a condition in a patient, which condition is characterised by the expression of one or more rearranged V, D or J gene segment sequence characteristics, said method comprising:

(i) spatially isolating on a solid support a library of individual template DNA molecules derived from a DNA sample comprising B and/or T cell DNA, which template DNA molecules have been generated such that said rearranged V, D or J gene segments are localised to the region of contiguous nucleotides at the 5′ and/or 3′ terminal ends of said template;

(ii) amplifying said spatially isolated template DNA molecules to generate clusters of amplicons wherein each cluster is generated from an individual spatially isolated template DNA molecule;

(iii) bidirectionally sequencing one or more amplicons of one or more clusters wherein the forward and reverse sequence reads of said amplicons do not provide a contiguous read across the full length of the amplicon;

(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read;
and wherein:
(1) said portion is not less than 75% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all the nucleic acid sequence results of (a) and the linker sequence is the same for all the nucleic acid sequence results of (b); and

(v) analysing the sequence result.

In another embodiment said DNA sample of interest comprises B and/or T cell DNA and said one or more target nucleotide sequences are one or more rearranged V, D or J gene segments.

In yet another embodiment said target nucleotide sequences are the DJ or VDJ rearrangements of IgH, TCR β or TCR δ or the VJ rearrangement of Igκ, Igλ, TCRα or TCRγ. In still another embodiment, said rearrangement is a kappa deleting element rearrangement.

In still yet another embodiment, said target nucleotide sequences are a V gene segment region, such as a region predisposed to undergoing hypermutation and/or a J gene segment region encoding a portion of the CDR3.

In yet still another embodiment, said target nucleotide sequences are the gene segment regions encoding all or some of the V leader sequence, the V region predisposed to somatic hypermutation, IgH FR1, IgH FR2 or IgH FR3.

In another embodiment, said contiguous nucleotide region of step (i) corresponds to about 80% of the maximum forward and reverse read length deliverable by the bidirectional sequencing technology selected for use in step (iii).

In a further embodiment said contiguous nucleotide region corresponds to 75%, 76%, 77%, 78%, 79%, 80%, 81%, 82% or 83% of the maximum forward and reverse read length deliverable by the bidirectional sequencing technology selected for use in step (iii) and said forward and reverse read portions is not less than 75%, 76%, 77%, 78%, 79%, 80%, 81%, 82% or 83% of the maximum forward and reverse read length deliverable by the bidirectional sequencing technology selected for use in step (iii).

In yet another embodiment, said target DNA sequences are localised to the 120 contiguous nucleotides at the 5′ and/or 3′ terminal ends of said template but wherein the 20 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites.

In yet still another embodiment, said target DNA sequences are localised to the 125 contiguous nucleotides at the 5′ and/or 3′ terminal ends of said template but wherein up to the 30 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites.

In another embodiment, said linker is 5-25 nucleotides in length. In still another embodiment said linker is 5-20 nucleotides in length. In a further embodiment, the length of said linker is 5, 6, 7, 8, 9, 10, 11, 12, 13, 14, 15 or 16 nucleotides, most preferably 9, 10, 11 or 12 nucleotides in length.

According to this embodiment, said analysis comprises aligning the nucleic acid sequence results generated in step (iv) and determining the expression of the target nucleic acid sequences of interest.

In yet another embodiment, said condition which is characterised by the expression of one or more rearranged V, D or J gene segment sequence characteristics is infection, transplantation, autoimmunity, immunodeficiency, neoplasia or any other condition characterised by T or B cell clonal expansion.

Said method is useful in the context of diagnosis, prognosis, classification, prediction of disease risk, detection of recurrence of disease, immune surveillance or monitoring prophylactic or therapeutic efficacy.

With respect to this aspect of the present invention, reference to “monitoring” should be understood as a reference to testing the subject for the presence or level of the subject clonal population of cells after initial diagnosis of the existence of said population. “Monitoring” includes reference to conducting both isolated one-off tests or a series of tests over a period of days, weeks, months or years. The tests may be conducted for any number of reasons including, but not limited to, predicting the likelihood that a mammal which is in remission will relapse, screening for minimal residual disease, monitoring the effectiveness of a treatment protocol, checking the status of a patient who is in remission, monitoring the progress of a condition prior to or subsequently to the application of a treatment regime, in order to assist in reaching a decision with respect to suitable treatment or in order to test new forms of treatment. The method of the present invention is therefore useful as both a clinical tool and a research tool.

Reference to a “neoplastic cell” should be understood as a reference to a cell exhibiting abnormal “growth”. The term “growth” should be understood in its broadest sense and includes reference to proliferation. In this regard, an example of abnormal cell growth is the uncontrolled proliferation of a cell. The uncontrolled proliferation of a lymphoid cell may lead to a population of cells which take the form of either a solid tumour or a single cell suspension (such as is observed, for example, in the blood of a leukemic patient). A neoplastic cell may be a benign cell or a malignant cell. In a preferred embodiment, the neoplastic cell is a malignant cell. In this regard, reference to a “neoplastic condition” is a reference to the existence of neoplastic cells in the subject mammal. Although “neoplastic lymphoid condition” includes reference to disease conditions which are characterised by reference to the presence of abnormally high numbers of neoplastic cells such as occurs in leukemias, lymphomas and myelomas, this phrase should also be understood to include reference to the circumstance where the number of neoplastic cells found in a mammal falls below the threshold which is usually regarded as demarcating the shift of a mammal from an evident disease state to a remission state or vice versa (the cell number which is present during remission is often referred to as the “minimal residual disease”). Still further, even where the number of neoplastic cells present in a mammal falls below the threshold detectable by the screening methods utilised prior to the advent of the present invention, the mammal is nevertheless regarded as exhibiting a “neoplastic condition”.

Disease conditions suitable for analysis in the context of this embodiment include any lymphoid neoplasias such as acute lymphoblastic leukaemia, acute lymphocytic leukaemia, acute myeloid leukemia, acute promyelocytic leukemia, chronic lymphocytic leukaemia, chronic myeloid leukemia, myeloproliferative neoplasms, such as myeloma, systemic mastocytosis, lymphoma and hairy cell leukemia.

In one particular embodiment, the method of the present invention is used to detect minimum residual disease in the context of lymphoid neoplasia.

In another embodiment non-neoplastic diseases characterised by clonal lymphoid expansion include infection, allergy, autoimmunity, transplant rejection, immunotherapy, polycythemia vera, myelodysplasia and leukocytosis, such as lymphocytic leucocytosis.

In accordance with all of the preceding aspects, in one embodiment said glass surface is a glass slide or a flow cell.

In another embodiment the terminal end of said contiguous nucleotide region expresses one or more nucleic acid sequences corresponding to indexes, barcodes, unique molecular identifiers, sequencing primer hybridisation sites and index sequencing primer hybridisation sites;

In yet another embodiment, said amplification is bridge amplification.

In a further embodiment said contiguous nucleotide region corresponds to 75%, 76%, 77%, 78%, 79%, 80%, 81%, 82% or 83% of the maximum forward and reverse read length deliverable by the bidirectional sequencing technology selected for use in step (iii) and said forward and reverse read portions is not less than 75%, 76%, 77%, 78%, 79%, 80%, 81%, 82% or 83% of the maximum forward and reverse read length deliverable by the bidirectional sequencing technology selected for use in step (iii).

In yet another embodiment, said target DNA sequences are localised to the 120 contiguous nucleotides at the 5′ and/or 3′ terminal ends of said template but wherein the 20 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites.

In yet still another embodiment, said target DNA sequences are localised to the 125 contiguous nucleotides at the 5′ and/or 3′ terminal ends of said template but wherein up to the 30 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites.

### Computer-Implemented Methods, Computer-Readable Storage Mediums and Devices

Some aspects of the disclosure are directed to computer-implemented methods, and computer-readable storage mediums and devices that implement a method for preparing nucleic acid sequence results for analysis from non-overlapping sequence reads for screening a nucleic acid sample of interest for the expression of one or more target nucleotide sequences.

The computer-implemented methods, and computer-readable storage mediums and devices described herein provide advantages over prior art methods by allowing analysis of non-overlapping sequence reads without the use of a reference sequence. The methods comprise identifying forward and reverse sequence reads from co-localised non-overlapping read sequences, trimming the identified forward and reverse sequence reads (i.e., taking a predefined length from a 5′ portion of the forward sequence reads and a predefined length from a 5′ portion of the reverse sequence reads) and then taping them together (keeping one set of sequence reads (forward or reverse) constant and taking a reverse complement of the other set) with a nucleic acid linker comprising a pre-defined number of Ns (N refers to any nucleotide (e.g., any one of A, G, T or C) in between. In some embodiments, the computer-implemented methods, and computer-readable storage mediums and devices described herein process millions to billions of sequence reads. In some embodiments, the computer-implemented methods, and computer-readable storage mediums and devices described herein process at least 1 million, 5 million, 10 million, 20 million, 30 million, 40 million, 50 million, 100 million, 250 million, 500 million, 1 billion, 5 billion, 10 billion or more sequence reads.

The term “memory” as used herein comprises program memory and working memory. The program memory may have one or more programs or software modules. The working memory stores data or information used by the CPU in executing the functionality described herein.

The term “processor” may include a single core processor, a multi-core processor, multiple processors located in a single device, or multiple processors in wired or wireless communication with each other and distributed over a network of devices, the Internet, or the cloud. Accordingly, as used herein, functions, features or instructions performed or configured to be performed by a “processor”, may include the performance of the functions, features or instructions by a single core processor, may include performance of the functions, features or instructions collectively or collaboratively by multiple cores of a multi-core processor, or may include performance of the functions, features or instructions collectively or collaboratively by multiple processors, where each processor or core is not required to perform every function, feature or instruction individually. The processor may be a CPU (central processing unit). The processor may comprise other types of processors such as a GPU (graphical processing unit). In other aspects of the disclosure, instead of or in addition to a CPU executing instructions that are programmed in the program memory, the processor may be an ASIC (application-specific integrated circuit), analog circuit or other functional logic, such as a FPGA (field-programmable gate array), PAL (Phase Alternating Line) or PLA (programmable logic array).

The CPU is configured to execute programs (also described herein as modules or instructions) stored in a program memory to perform the functionality described herein. The memory may be, but not limited to, RAM (random access memory), ROM (read-only memory) and persistent storage. The memory is any piece of hardware that is capable of storing information, such as, for example without limitation, data, programs, instructions, program code, and/or other suitable information, either on a temporary basis and/or a permanent basis.

Various aspects of the present disclosure may be embodied as a program, software, or computer instructions embodied or stored in a computer or machine usable or readable medium, or a group of media which causes the computer or machine to perform the steps of the method when executed on the computer, processor, and/or machine. A program storage device readable by a machine, e.g., a computer readable medium, tangibly embodying a program of instructions executable by the machine to perform various functionalities and methods described in the present disclosure is also provided.

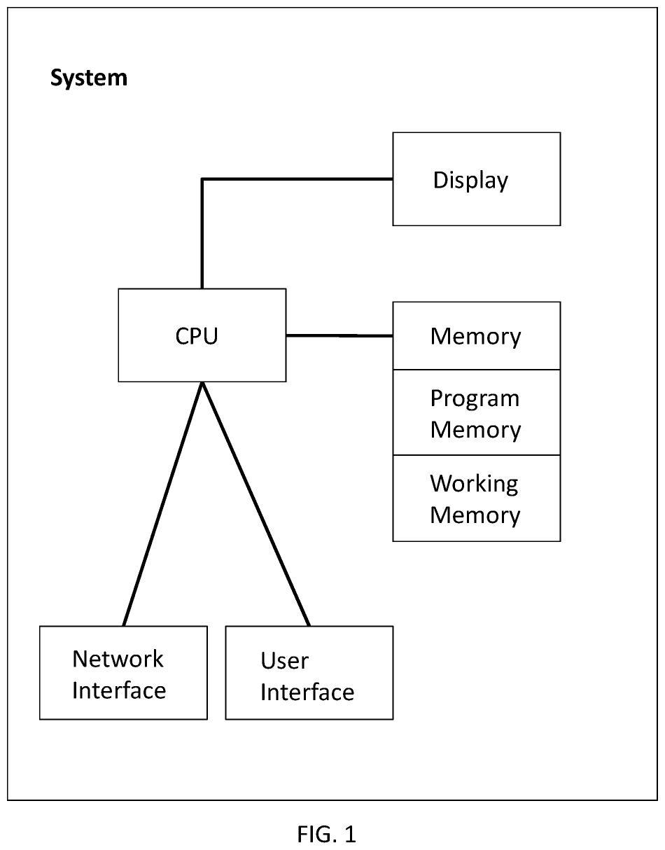

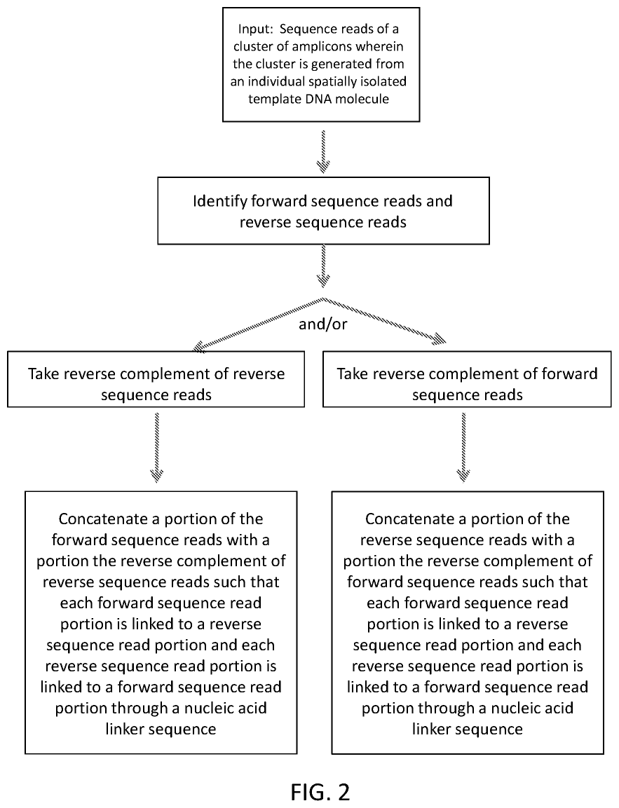

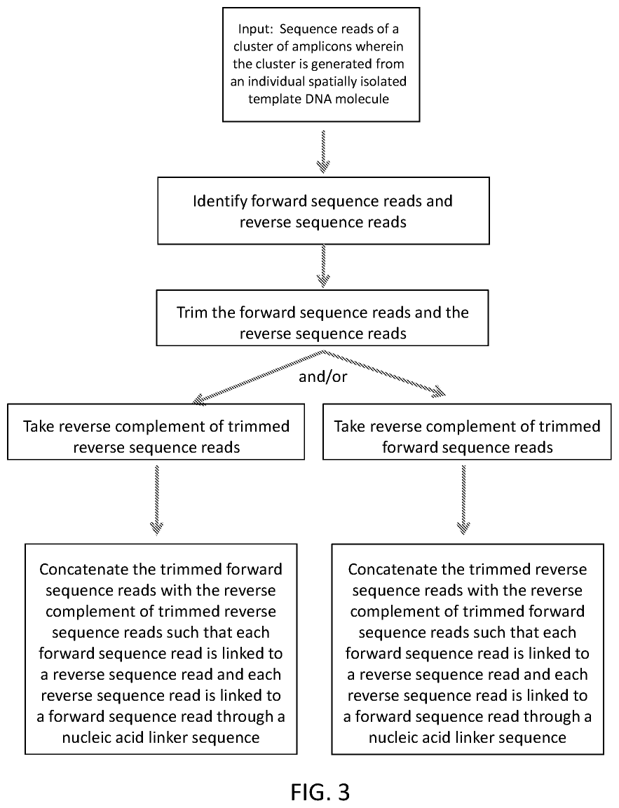

In some embodiments, the present disclosure includes a system comprising a CPU, a display, a network interface, a user interface, a memory, a program memory and a working memory (FIG. 1), where the system is programmed to execute a program, software, or computer instructions directed to methods or processes of the instant disclosure. Exemplary and non-limiting embodiments are shown in FIG. 2 and FIG. 3.

### Computer-Implemented Methods

An aspect of the disclosure is directed to a computer-implemented method for preparing nucleic acid sequence results for analysis from non-overlapping sequence reads from a cluster of amplicons.

In some embodiments, the computer-implemented method comprises identifying forward sequence reads and reverse sequence reads from sequence reads of the cluster of amplicons. In some embodiments, the forward and the reverse sequence reads are DNA sequence reads.

In some embodiments, a cluster of amplicons is generated from an individual spatially isolated template DNA molecule, and each sequence read is generated by a selected bidirectional sequencing technology. In some embodiments, the bidirectional sequencing technology is selected from the technologies listed in Table 1. In some embodiments, the forward sequence reads and the reverse sequence reads do not overlap and do not provide a contiguous read across the full length of any amplicon.

In some embodiments, the cluster of amplicons is amplified from B and/or T cell DNA. In some embodiments, the cluster of amplicons comprises at least one rearranged V, D or J gene segment. In some embodiments, the cluster of amplicons comprises DJ or VDJ rearrangements of IgH, TCR β or TCR δ or the VJ rearrangement of Igκ, Igλ, TCRα or TCRγ. In a specific embodiment, the VJ rearrangement is a kappa deleting element rearrangement. In some embodiments, the cluster of amplicons comprises a V gene segment region, such as a region predisposed to undergoing hypermutation and/or a J gene segment region encoding a portion of the CDR3. In some embodiments, the cluster of amplicons comprises gene segment regions encoding all or some of the V leader sequence, the V region predisposed to somatic hypermutation. IgH FR1, IgH FR2 or IgH FR3.

In some embodiments, the computer-implemented method comprises linking the forward sequence reads with the reverse sequence reads resulting in a plurality of first nucleic acid sequence results, such that each forward sequence read is linked to a reverse sequence read and each reverse sequence read is linked to a forward sequence read through a first nucleic acid linker sequence.

In some embodiments, each linking is achieved by: concatenating the first nucleic acid linker sequence between the 3′ end of a portion of the terminal 5′ contiguous nucleic acid sequence of a forward sequence read and the reverse complement of a portion of the terminal 5′ contiguous nucleic acid sequence of a reverse sequence read, thereby producing a first nucleic acid sequence result comprising the portion of the forward sequence read, the first nucleic acid linker sequence, and the reverse complement of the portion of the reverse sequence read in that order.

In some embodiments, the identifying is achieved by one or more indexes, barcodes, unique molecular identifiers, sequencing primer hybridisation sites or index sequencing primer hybridisation sites that are found on forward sequence reads and reverse sequence reads, wherein the one or more indexes, barcodes, unique molecular identifiers, sequencing primer hybridisation sites or index sequencing primer hybridisation sites found on forward sequence reads are different from the one or more indexes, barcodes, unique molecular identifiers, sequencing primer hybridisation sites or index sequencing primer hybridisation sites found on reverse sequence reads.

In some embodiments, the computer-implemented method further comprises linking the forward sequence reads with the reverse sequence reads resulting in a plurality of second nucleic acid sequence results, such that each forward sequence read is linked to a reverse sequence read and each reverse sequence read is linked to a forward sequence read through a second nucleic acid linker sequence, wherein each linking is achieved by concatenating the second nucleic acid linker sequence between the 3′ end of a portion of the terminal 5′ contiguous nucleic acid sequence of a reverse sequence read and the reverse complement of a portion of the terminal 5′ contiguous nucleic acid sequence of a forward sequence read, thereby producing a second nucleic acid sequence result comprising the portion from the reverse sequence read, the second nucleic acid linker sequence and the reverse complement of the portion from the forward sequence read in that order, wherein (1) the length of the portion from the forward sequence read is not less than 75% of the maximum read length deliverable by the selected bidirectional sequencing technology, the length of the portion from the reverse sequence read is not less than 75% of the maximum read length deliverable by the selected bidirectional sequencing technology; (2) the length of the portion from the reverse sequence read being concatenated to the second nucleic acid linker is the same for all reverse sequence reads and is the same as the length of the portion from the reverse sequence read being concatenated to the first nucleic acid linker, (3) the length of the portion from the forward sequence read being concatenated to the second nucleic acid linker is the same for all forward sequence reads and is the same as the length of the portion from the forward sequence read being concatenated to the first nucleic acid linker, but may be the same or different to the length of the portion from the reverse sequence read being concatenated to the second nucleic acid linker; and (4) the second nucleic acid linker sequence is the same for all second nucleic acid sequence results.

In some embodiments, the length of the portion from the forward sequence read is not less than about 75%, 76%, 77%, 78%, 79%, 80%, 81%, 82% or 83% of the maximum read length deliverable by the selected bidirectional sequencing technology, the length of the portion from the reverse sequence read is not less than about 75%, 76%, 77%, 78%, 79%, 80%, 81%, 82% or 83% of the maximum read length deliverable by the selected bidirectional sequencing technology. In some embodiments, the length of the portion from the reverse sequence read is the same for all reverse sequence reads which are analysed. In some embodiments, the length of the portion from the forward sequence read is the same for all forward sequence reads which are analysed but may be the same or different to the length of the portion from the reverse sequence read. In some embodiments, the length of the portion of the forward sequence read is the same as the length of the portion of the reverse sequence read.

In some embodiments, the portion of the forward sequence read comprises a specified number of contiguous nucleotides of the 5′ terminus of the forward sequence read, and the portion of the reverse sequence read comprises a specified number of contiguous nucleotides of the 5′ terminus of the reverse sequence read. In some embodiments, the specified number of contiguous nucleotides comprises between about 80 nucleotides and about 180 nucleotides. As used in this disclosure, the term “about” refers to ±10% of a given value. In some embodiments, the specified number of contiguous nucleotides comprises about, 80, about 90, about 100, about 110, about 120, about 130, about 140, about 150, about 160, about 170, or about 180 nucleotides.

In some embodiments, the first nucleic acid linker sequence is the same for all first nucleic acid sequence results. In some embodiments, the first nucleic acid linker sequence is between 5-30 nucleotides in length, between 5-25 nucleotides in length or between 5-20 nucleotides in length. In some embodiments, the length of the first nucleic acid linker sequence is at least 5, 6, 7, 8, 9, 10, 11, 12, 13, 14, 15 or 16 nucleotides long.

In some embodiments, the first nucleic acid linker sequence and the second nucleic acid linker sequence are at least 11 nucleotides long. In some embodiments, the first nucleic acid linker sequence and the second nucleic acid linker sequence are between 5-30 nucleotides in length, between 5-25 nucleotides in length or between 5-20 nucleotides in length. In some embodiments, the length of the first nucleic acid linker sequence is at least 5, 6, 7, 8, 9, 10, 11, 12, 13, 14, 15 or 16 nucleotides long. In some embodiments, the length of the second nucleic acid linker sequence is at least 5, 6, 7, 8, 9, 10, 11, 12, 13, 14, 15 or 16 nucleotides long.

### Computer-Readable Storage Medium

An aspect of the disclosure is directed to a non-transitory computer-readable storage medium having program instructions embodied therewith, the program instructions executable by a processing element of a device to cause the device to implement a method for preparing nucleic acid sequence results for analysis from non-overlapping sequence reads from a cluster of amplicons.

In some embodiments, the non-transitory computer-readable storage medium comprises instructions for identifying forward sequence reads and reverse sequence reads from sequence reads of the cluster of amplicons. In some embodiments, the forward and the reverse sequence reads are DNA sequence reads.

In some embodiments, a cluster of amplicons is generated from an individual spatially isolated template DNA molecule, and each sequence read is generated by a selected bidirectional sequencing technology. In some embodiments, the bidirectional sequencing technology is selected from the technologies listed in Table 1. In some embodiments, the forward sequence reads and the reverse sequence reads do not overlap and do not provide a contiguous read across the full length of any amplicon.

In some embodiments, the cluster of amplicons is amplified from B and/or T cell DNA. In some embodiments, the cluster of amplicons comprises at least one rearranged V, D or J gene segment. In some embodiments, the cluster of amplicons comprises DJ or VDJ rearrangements of IgH, TCR β or TCR δ or the VJ rearrangement of Igκ, Igλ, TCRα or TCRγ. In a specific embodiment, the VJ rearrangement is a kappa deleting element rearrangement. In some embodiments, the cluster of amplicons comprises a V gene segment region, such as a region predisposed to undergoing hypermutation and/or a J gene segment region encoding a portion of the CDR3. In some embodiments, the cluster of amplicons comprises gene segment regions encoding all or some of the V leader sequence, the V region predisposed to somatic hypermutation, IgH FR1, IgH FR2 or IgH FR3.

In some embodiments, the non-transitory computer-readable storage medium comprises instructions for linking the forward sequence reads with the reverse sequence reads resulting in a plurality of first nucleic acid sequence results, such that each forward sequence read is linked to a reverse sequence read and each reverse sequence read is linked to a forward sequence read through a first nucleic acid linker sequence.

In some embodiments, each linking is achieved by: concatenating the first nucleic acid linker sequence between the 3′ end of a portion of the terminal 5′ contiguous nucleic acid sequence of a forward sequence read and the reverse complement of a portion of the terminal 5′ contiguous nucleic acid sequence of a reverse sequence read, thereby producing a first nucleic acid sequence result comprising the portion of the forward sequence read, the first nucleic acid linker sequence, and the reverse complement of the portion of the reverse sequence read in that order.

In some embodiments, the non-transitory computer-readable storage medium comprises further instructions for linking the forward sequence reads with the reverse sequence reads resulting in a plurality of second nucleic acid sequence results, such that each forward sequence read is linked to a reverse sequence read and each reverse sequence read is linked to a forward sequence read through a second nucleic acid linker sequence, wherein each linking is achieved by concatenating the second nucleic acid linker sequence between the 3′ end of a portion of the terminal 5′ contiguous nucleic acid sequence of a reverse sequence read and the reverse complement of a portion of the terminal 5′ contiguous nucleic acid sequence of a forward sequence read, thereby producing a second nucleic acid sequence result comprising the portion from the reverse sequence read, the second nucleic acid linker sequence and the reverse complement of the portion from the forward sequence read in that order; wherein (1) the length of the portion from the forward sequence read is not less than 75% of the maximum read length deliverable by the selected bidirectional sequencing technology, the length of the portion from the reverse sequence read is not less than 75% of the maximum read length deliverable by the selected bidirectional sequencing technology; (2) the length of the portion from the reverse sequence read being concatenated to the second nucleic acid linker is the same for all reverse sequence reads and is the same as the length of the portion from the reverse sequence read being concatenated to the first nucleic acid linker; (3) the length of the portion from the forward sequence read being concatenated to the second nucleic acid linker is the same for all forward sequence reads and is the same as the length of the portion from the forward sequence read being concatenated to the first nucleic acid linker, but may be the same or different to the length of the portion from the reverse sequence read being concatenated to the second nucleic acid linker; and (4) the second nucleic acid linker sequence is the same for all second nucleic acid sequence results.

In some embodiments, the identifying is achieved by one or more indexes, barcodes, unique molecular identifiers, sequencing primer hybridisation sites or index sequencing primer hybridisation sites that are found on forward sequence reads and reverse sequence reads, wherein the one or more indexes, barcodes, unique molecular identifiers, sequencing primer hybridisation sites or index sequencing primer hybridisation sites found on forward sequence reads are different from the one or more indexes, barcodes, unique molecular identifiers, sequencing primer hybridisation sites or index sequencing primer hybridisation sites found on reverse sequence reads.

In some embodiments, the identifying is achieved by one or more indexes, barcodes, unique molecular identifiers, sequencing primer hybridisation sites or index sequencing primer hybridisation sites that are found on forward sequence reads and reverse sequence reads, wherein the one or more indexes, barcodes, unique molecular identifiers, sequencing primer hybridisation sites or index sequencing primer hybridisation sites found on forward sequence reads are different from the one or more indexes, barcodes, unique molecular identifiers, sequencing primer hybridisation sites or index sequencing primer hybridisation sites found on reverse sequence reads.

In some embodiments, the length of the portion from the forward sequence read is not less than about 75%, 76%, 77%, 78%, 79%, 80%, 81%, 82% or 83% of the maximum read length deliverable by the selected bidirectional sequencing technology, the length of the portion from the reverse sequence read is not less than about 75%, 76%, 77%, 78%, 79%, 80%, 81%, 82% or 83% of the maximum read length deliverable by the selected bidirectional sequencing technology. In some embodiments, the length of the portion from the reverse sequence read is the same for all reverse sequence reads which are analysed. In some embodiments, the length of the portion from the forward sequence read is the same for all forward sequence reads which are analysed but may be the same or different to the length of the portion from the reverse sequence read. In some embodiments, the length of the portion of the forward sequence read is the same as the length of the portion of the reverse sequence read.

In some embodiments, the portion of the forward sequence read comprises a specified number of contiguous nucleotides of the 5′ terminus of the forward sequence read, and the portion of the reverse sequence read comprises a specified number of contiguous nucleotides of the 5′ terminus of the reverse sequence read. In some embodiments, the specified number of contiguous nucleotides comprises between about 80 nucleotides and about 180 nucleotides. As used in this disclosure, the term “about” refers to ±10% of a given value. In some embodiments, the specified number of contiguous nucleotides comprises about 80, about 90, about 100, about 110, about 120, about 130, about 140, about 150, about 160, about 170, or about 180 nucleotides.

In some embodiments, the first nucleic acid linker sequence is the same for all first nucleic acid sequence results. In some embodiments, the first nucleic acid linker sequence is between 5-30 nucleotides in length, between 5-25 nucleotides in length or between 5-20 nucleotides in length. In some embodiments, the length of the first nucleic acid linker sequence is at least 5, 6, 7, 8, 9, 10, 11, 12, 13, 14, 15 or 16 nucleotides long.

In some embodiments, the first nucleic acid linker sequence and the second nucleic acid linker sequence are at least 11 nucleotides long. In some embodiments, the first nucleic acid linker sequence and the second nucleic acid linker sequence are between 5-30 nucleotides in length, between 5-25 nucleotides in length or between 5-20 nucleotides in length. In some embodiments, the length of the first nucleic acid linker sequence is at least 5, 6, 7, 8, 9, 10, 11, 12, 13, 14, 15 or 16 nucleotides long. In some embodiments, the length of the second nucleic acid linker sequence is at least 5, 6, 7, 8, 9, 10, 11, 12, 13, 14, 15 or 16 nucleotides long.

### Device

Another aspect of the disclosure is directed to a device for preparing nucleic acid sequence results for analysis from non-overlapping sequence reads. The device comprises a hardware processor that is configured to identify forward sequence reads and reverse sequence reads from sequence reads of a cluster of amplicons.

In some embodiments, the hardware processor configured for identifying forward sequence reads and reverse sequence reads from sequence reads of the cluster of amplicons. In some embodiments, the forward and the reverse sequence reads are DNA sequence reads.

In some embodiments, the hardware processor configured for linking the forward sequence reads with the reverse sequence reads resulting in a plurality of first nucleic acid sequence results, such that each forward sequence read is linked to a reverse sequence read and each reverse sequence read is linked to a forward sequence read through a first nucleic acid linker sequence.

In some embodiments, each linking is achieved by: concatenating the first nucleic acid linker sequence between the 3′ end of a portion of the terminal 5′ contiguous nucleic acid sequence of a forward sequence read and the reverse complement of a portion of the terminal 5′ contiguous nucleic acid sequence of a reverse sequence read, thereby producing a first nucleic acid sequence result comprising the portion of the forward sequence read, the first nucleic acid linker sequence, and the reverse complement of the portion of the reverse sequence read in that order.

In some embodiments, a cluster of amplicons is generated from an individual spatially isolated template DNA molecule, and each sequence read is generated by a selected bidirectional sequencing technology. In some embodiments, the bidirectional sequencing technology is selected from the technologies listed in Table 1. In some embodiments, the forward sequence reads and the reverse sequence reads do not overlap and do not provide a contiguous read across the full length of any amplicon.

In some embodiments, the cluster of amplicons is amplified from B and/or T cell DNA. In some embodiments, the cluster of amplicons comprises at least one rearranged V, D or J gene segment. In some embodiments, the cluster of amplicons comprises DJ or VDJ rearrangements of IgH, TCR β or TCR δ or the VJ rearrangement of Igκ, Igλ, TCRα or TCRγ. In a specific embodiment, the VJ rearrangement is a kappa deleting element rearrangement. In some embodiments, the cluster of amplicons comprises a V gene segment region, such as a region predisposed to undergoing hypermutation and/or a J gene segment region encoding a portion of the CDR3. In some embodiments, the cluster of amplicons comprises gene segment regions encoding all or some of the V leader sequence, the V region predisposed to somatic hypermutation, IgH FR1, IgH FR2 or IgH FR3.

In some embodiments, the non-transitory computer-readable storage medium comprises further instructions for linking the forward sequence reads with the reverse sequence reads resulting in a plurality of second nucleic acid sequence results, such that each forward sequence read is linked to a reverse sequence read and each reverse sequence read is linked to a forward sequence read through a second nucleic acid linker sequence, wherein each linking is achieved by concatenating the second nucleic acid linker sequence between the 3′ end of a portion of the terminal 5′ contiguous nucleic acid sequence of a reverse sequence read and the reverse complement of a portion of the terminal 5′ contiguous nucleic acid sequence of a forward sequence read, thereby producing a second nucleic acid sequence result comprising the portion from the reverse sequence read, the second nucleic acid linker sequence and the reverse complement of the portion from the forward sequence read in that order, wherein (1) the length of the portion from the forward sequence read is not less than 75% of the maximum read length deliverable by the selected bidirectional sequencing technology, the length of the portion from the reverse sequence read is not less than 75% of the maximum read length deliverable by the selected bidirectional sequencing technology; (2) the length of the portion from the reverse sequence read being concatenated to the second nucleic acid linker is the same for all reverse sequence reads and is the same as the length of the portion from the reverse sequence read being concatenated to the first nucleic acid linker; (3) the length of the portion from the forward sequence read being concatenated to the second nucleic acid linker is the same for all forward sequence reads and is the same as the length of the portion from the forward sequence read being concatenated to the first nucleic acid linker, but may be the same or different to the length of the portion from the reverse sequence read being concatenated to the second nucleic acid linker; and (4) the second nucleic acid linker sequence is the same for all second nucleic acid sequence results.

In some embodiments, the length of the portion from the forward sequence read is not less than about 75%, 76%, 77%, 78%, 79%, 80%, 81%, 82% or 83% of the maximum read length deliverable by the selected bidirectional sequencing technology, the length of the portion from the reverse sequence read is not less than about 75%, 76%, 77%, 78%, 79%, 80%, 81%, 82% or 83% of the maximum read length deliverable by the selected bidirectional sequencing technology. In some embodiments, the length of the portion from the reverse sequence read is the same for all reverse sequence reads which are analysed. In some embodiments, the length of the portion from the forward sequence read is the same for all forward sequence reads which are analysed but may be the same or different to the length of the portion from the reverse sequence read. In some embodiments, the length of the portion of the forward sequence read is the same as the length of the portion of the reverse sequence read.

In some embodiments, the portion of the forward sequence read comprises a specified number of contiguous nucleotides of the 5′ terminus of the forward sequence read, and the portion of the reverse sequence read comprises a specified number of contiguous nucleotides of the 5′ terminus of the reverse sequence read. In some embodiments, the specified number of contiguous nucleotides comprises between about 80 nucleotides and about 180 nucleotides. As used in this disclosure, the term “about” refers to 10% of a given value. In some embodiments, the specified number of contiguous nucleotides comprises about 80, about 90, about 100, about 110, about 120, about 130, about 140, about 150, about 160, about 170, or about 180 nucleotides.

In some embodiments, the first nucleic acid linker sequence is the same for all first nucleic acid sequence results. In some embodiments, the first nucleic acid linker sequence is between 5-30 nucleotides in length, between 5-25 nucleotides in length or between 5-20 nucleotides in length. In some embodiments, the length of the first nucleic acid linker sequence is at least 5, 6, 7, 8, 9, 10, 11, 12, 13, 14, 15 or 16 nucleotides long.

In some embodiments, the first nucleic acid linker sequence and the second nucleic acid linker sequence are at least 11 nucleotides long. In some embodiments, the first nucleic acid linker sequence and the second nucleic acid linker sequence are between 5-30 nucleotides in length, between 5-25 nucleotides in length or between 5-20 nucleotides in length. In some embodiments, the length of the first nucleic acid linker sequence is at least 5, 6, 7, 8, 9, 10, 11, 12, 13, 14, 15 or 16 nucleotides long. In some embodiments, the length of the second nucleic acid linker sequence is at least 5, 6, 7, 8, 9, 10, 11, 12, 13, 14, 15 or 16 nucleotides long.

Further features of the present invention are more fully described in the following non-limiting examples.

### Example 1

### Methods

Paired-end sequencing is a standard tool for analyzing B-cell or T-cell clonality. When the sequencing length is sufficient, an entire rearrangement can be sequenced by utilizing the overlap between the two reads in a pair. This “complete” sequencing allows for straight-forward analysis without any additional formatting steps. If sequencing length is insufficient (for reasons of platform limitations or assay design, for example), the analysis used in the “complete” sequencing scenario becomes prone to errors. Described herein is a method for analyzing non-overlapping sequencing data for the purpose of clonality assessment.

The analysis method for “complete” sequencing (where the paired-reads overlap each other and the entire sequence of the amplicon can be identified) begins with identifying the overlap and producing a concatenated sequence comprising the unique, non-overlapping sequence of read 1 (R1), followed by the overlapping sequence between read 1 and read 2 (R1 and R2), and culminating in the unique, non-overlapping sequence of read 2 (R2). When the sequencing platform/assay does not support generating an overlapping sequence, the following modifications allow for downstream analysis to occur.

Simple Taping: The simplest method is to “tape” the read pair (R1 and R2) together with a unique sequence in between. Because the downstream analysis involves alignment to a reference, it is important to use a sequence that cannot be involved with this alignment step. A sequence of 11 “N” is chosen (11-Nmer), as such a sequence will generally not be aligned by standard alignment algorithm practices (not attempting to align “Ns” as they are considered unknown nucleotides). First, the R2 read is reverse complemented (rcR2) to be in the sense orientation to R1. Then the 11-Nmer is concatenated to the end of R1. Finally, the R2 read is concatenated to the end of the R1+11-Nmer sequence, producing a R1+11-Nmer+rcR2 read. This concatenated read is now ready for downstream analysis.

Smart Taping: “Smart Taping” is similar to the Simple Taping method, except the read pairs are modified before concatenation to the 11-Nmer. The R1 and R2 reads are first identified by which gene specific primers amplified these reads, which is simply down by looking at the initial 20-25 nts of sequence and matching it with the known primer sequences. From the end of the primer sequence (i.e. an anchor point), an additional 100 nts are saved, and the remaining sequence is removed (for both the R1 and R2 reads), resulting in “trimmed” R1 and R2 reads. At this point, the trimmed reads are treated in the same way as the Simple Taping method: trimmed R2 is reverse complemented, and the 11-Nmer is concatenated to the trimmed R1, and the trimmed rcR2 is concatenated to the trimmed R1+11-Nmer. This concatenated trimmed read is now ready for downstream analysis.

Downstream Analysis: Briefly, identical reads are collapsed into single entries with a counter attached to their header to annotate how many copies existed in the dataset. The collapsed reads are aligned to a reference and assigned a V-gene and J-gene based on best alignment, and quantitative information is output regarding the total counts and relative frequency of each read.

### Example 2

### MiSeq Paired-End Sequencing

Dataset: A MiSeq sequencing run (2×251 cycles) consisting of a 10% contrived cell line DNA diluted in tonsil background DNA was used for demonstration of the taping method efficiency. While the 2×251 cycle run allows for a “complete” sequencing analysis of the chosen target (LymphoTrack IGH FR1 assay), the data contained within this run was truncated to mimic 2×151 cycles by removing the last 100 nts of every read contained within the R1 and R2 paired files. The 2×251 cycle data will be called the “control” dataset, while the truncated 2.151 cycle data will be called the “tape test” dataset.

Additionally, a Nextseq sequencing run (2×151 cycles) consisting of 100% cell line DNA was used for demonstrating a real-world use case of taping method efficiency.

Results

MiSeq Control Dataset Results Using Complete Sequencing: The control dataset was analyzed using the “complete” analysis, consisting of overlapping the paired reads before doing the downstream analysis. The results are contained in Table 2.

TABLE 2
Control Dataset Results

| Rank | Length | Raw count | V-gene | J-gene | % total reads |
|---|---|---|---|---|---|
| 1 | 280 | 86653 | IGHV3-9_01 | IGHJ4_02 | 9.466414 |
| 2 | 282 | 1517 | IGHV3-9_01 | IGHJ4_02 | 0.165725 |
| 3 | 280 | 1092 | IGHV3-9_01 | IGHJ4_02 | 0.119296 |
| 4 | 283 | 363 | IGHV1-18_01 | IGHJ6_02 | 0.039656 |
| 5 | 262 | 360 | IGHV1-3_01 | IGHJ4_02 | 0.039328 |

This is the expected result for this 10% contrived dataset using a “complete” sequencing platform/assay, with the V3-J4 rearrangement being found near the 10% frequency (9.45% here).

MiSeq Tape Test Dataset Results Using Simple Taping: The MiSeq tape test dataset was analyzed using the “simple tape” analysis, consisting of adding an 11-Nmer sequence in between the R1 and R2 reads. The results are contained in Table 3.

TABLE 3
MiSeq Tape Test Dataset, Simple Taping Results

| Rank | Length | Raw count | V-gene | J-gene | % total reads |
|---|---|---|---|---|---|
| 1 | 259 | 50558 | IGHV3-9_01 | IGHJ4_02 | 5.235579 |
| 2 | 258 | 36314 | IGHV3-9_01 | IGHJ4_02 | 3.760529 |
| 3 | 257 | 1405 | IGHV3-9_01 | IGHJ4_02 | 0.145496 |
| 4 | 259 | 1375 | IGHV3-9_01 | IGHJ4_02 | 0.142389 |
| 5 | 258 | 1018 | IGHV3-9_01 | IGHJ4_02 | 0.10542 |

The results show that the simple taping method results in the 10% clonal sequence being split into multiple sequences of differing lengths. The reason for this seems to arise from the choice of where to place the 11-Nmer during the taping step. Below is an alignment of the upstream and downstream regions of the 11-Nmer for these top 5 reads, with dashes representing gaps in the alignment of sequence not present in the read. Reads rank 2 and 5 have a single gap, while read rank 3 has 4 nts of gap.

Rank 1
(SEQ ID NO: 1)
GCTATGCGGACTCTGNNNNNNNNNNNGCCAAGAACTC
Rank 2
(SEQ ID NO: 2)
GCTATGCGGACTCT-NNNNNNNNNNNGCCAAGAACTC
Rank 3
(SEQ ID NO: 3)
GCTATGCGGAC----NNNNNNNNNNNGCCAAGAACTC
Rank 4
(SEQ ID NO: 4)
GCTATGCGGACTCTGNNNNNNNNNNNGCCAAGAACTC
Rank 5
(SEQ ID NO: 5)
GCTATGCGGACTCT-NNNNNNNNNNNGCCAAGAACTC

During the simple taping step, the 11-Nmer is concatenated directly to the end of the R1 read. Closer inspection of the taping region shows that the end of the R1 read does not consistently end in the same position for reads that are supposed to be the same sequence. This phenomenon has a demonstrably negative result in reducing the top read signal, notably because the sequence of the reads is no longer identical and are not collapsed during the downstream analysis.

MiSeq Tape Test Dataset Results Using Smart Taping: The MiSeq tape test dataset was then analyzed using the smart taping method, which trims off sequence from the R1 and R2 reads that are 100 nts or more away from the primer site. The results are found in Table 4.

TABLE 4
MiSeq Tape Test Dataset, Smart Taping Results

| Rank | Length | Raw count | V-gene | J-gene | % total reads |
|---|---|---|---|---|---|
| 1 | 208 | 95849 | IGHV3-9_01 | IGHJ4_02 | 9.976934 |
| 2 | 208 | 2588 | IGHV3-9_01 | IGHJ4_02 | 0.269385 |
| 3 | 208 | 1737 | IGHV3-9_01 | IGHJ4_02 | 0.180805 |
| 4 | 208 | 1226 | IGHV3-9_01 | IGHJ4_02 | 0.127615 |
| 5 | 208 | 553 | IGHV4-34_11 | IGHJ6_02 | 0.057562 |

The results show that reducing the sequence length by using an anchor point to trim off the “fuzzy” end of the reads can restore the expected ratio as measured by the complete sequencing approach.

### Example 3

### Nextseq Paired-End Sequencing

NextSeq Tape Test Dataset Results Using Simple Taping: The NextSeq tape test dataset was analyzed using the “simple tape” analysis, consisting of adding an 11-Nmer sequence in between the R1 and R2 reads. The results are contained in Table 5.

TABLE 5
NextSeq Tape Test Dataset, Simple Taping Results

| Rank | Length | Raw count | V-gene | J-gene | % total reads |
|---|---|---|---|---|---|
| 1 | 243 | 10050 | IGHV2-70_13 | IGHJ6_02 | 14.04239 |
| 2 | 257 | 2725 | IGHV2-70_13 | IGHJ6_02 | 3.807514 |
| 3 | 244 | 2688 | IGHV2-70_13 | IGHJ6_02 | 3.755816 |
| 4 | 258 | 2565 | IGHV2-70_13 | IGHJ6_02 | 3.583954 |
| 5 | 257 | 2092 | IGHV2-70_13 | IGHJ6_02 | 2.923053 |

The results show that the simple taping method results in the 100% clonal sequence being split into multiple sequences of differing lengths. The reason for this seems to arise from the choice of where to place the 11-Nmer during the taping step. Below is an alignment of the upstream and downstream regions of the 11-Nmer for these top 5 reads, with dashes representing gaps in the alignment of sequence not present in the read. Reads rank 1 has a single gap, rank 2 and 5 have a triple gap, rank 3 has no gap, and rank 4 has a double gap.

Rank 1
(SEQ ID NO: 6)
GATTGGGATGNNNNNNNNNNNNN-CCAGGTGGT
Rank 2
(SEQ ID NO: 7)
GATTGGGATGNNNNNNNNNNNNN---AGGTGGT
Rank 3
(SEQ ID NO: 8)
GATTGGGATGNNNNNNNNNNNNNACCAGGTGGT
Rank 4
(SEQ ID NO: 9)
GATTGGGATGNNNNNNNNNNNNN--CAGGTGGT
Rank 5
(SEQ ID NO: 10)
GATTGGGATGNNNNNNNNNNNNN---AGGTGGT

During the simple taping step, the 11-Nmer is concatenated directly to the end of the R1 read and the beginning of the rcR2. Closer inspection of the taping region shows that the beginning of the rcR2 read (which is also the end of the R2 read) does not consistently start in the same position for reads that are supposed to be the same sequence. This phenomenon has a demonstrably negative result in reducing the top read signal, notably because the sequence of the reads is no longer identical and are not collapsed during the downstream analysis.

NextSeq Tape Test Dataset Results Using Smart Taping: The NextSeq tape test dataset was then analyzed using the smart taping method, which trims off sequence from the R1 and R2 reads that are 100 nts or more away from the primer site. The results are found in Table 6.

TABLE 6
NextSeq Tape Test Dataset, Smart Taping Results

| Rank | Length | Raw count | V-gene | J-gene | % total reads |
|---|---|---|---|---|---|
| 1 | 208 | 4662164 | IGHV2-70_13 | IGHJ6_02 | 37.52596 |
| 2 | 208 | 823364 | IGHV2-70_13 | IGHJ6_02 | 6.627292 |
| 3 | 208 | 768631 | IGHV3-9_01 | IGHJ4_02 | 6.186744 |
| 4 | 208 | 62747 | IGHV3-9_01 | IGHJ4_02 | 0.505053 |
| 5 | 208 | 48764 | IGHV3-9_01 | IGHJ4_02 | 0.392504 |

The results show that reducing the sequence length by using an anchor point to trim off the “fuzzy” ends of the reads can greatly improve the signal captured.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

1. A method of screening a nucleic acid sample of interest for the expression of one or more target nucleotide sequences, said method comprising:
(i) spatially isolating on a solid support a library of individual template DNA molecules derived from said nucleic acid sample, which template DNA molecules have been generated such that the target nucleotide sequences are localised to the region of contiguous nucleotides at the 5′ and/or 3′ terminal ends of said template;
(ii) amplifying said spatially isolated template DNA molecules to generate clusters of amplicons wherein each cluster is generated from an individual spatially isolated template DNA molecule;
(iii) bidirectionally sequencing one or more amplicons of one or more clusters wherein the forward and reverse sequence reads of said amplicons do not provide a contiguous read across the full length of the amplicon;
(iv) identifying the forward and reverse sequence reads for the one or more clusters which are sequenced in accordance with step (iii) and generating a nucleic acid sequence result comprising:
(a) a portion of the terminal 5′ contiguous nucleic acid sequence of the forward read which is linked at its 3′ end to one of the terminal ends of a nucleic acid linker sequence and which linker sequence is linked at its other terminal end to the sequence complementary to a portion of the terminal 5′ contiguous nucleic acid sequence of the reverse read; and/or
(b) a portion of the terminal 5′ contiguous nucleic acid sequence of the reverse read which is linked at its 3′ end to one of the terminal ends of a nucleic acid linker sequence and which linker sequence is linked at its other terminal end to the sequence complementary to a portion of the terminal 5′ contiguous nucleic acid sequence of the forward read;
and wherein:
(1) said portion is not less than 75% of the maximum forward and reverse read length deliverable by the selected bidirectional sequencing technology, (2) said portion of the reverse read contiguous sequence is the same for all reverse reads which are analysed, (3) said portion of the forward read contiguous sequence is the same for all forward reads which are analysed but may be the same or different to the reverse read portion and (4) the linker sequence is the same for all the nucleic acid sequence results of (a) and the linker sequence is the same for all the nucleic acid sequence results of (b); and
(v) analysing the sequence result.

2. The method according to claim 1, wherein said method further comprises diagnosing, monitoring or otherwise screening for a condition in a patient, which condition is characterised by the expression of one or more target nucleotide sequences.

3. (canceled)

4. The method according to claim 1 wherein said nucleic sample of interest comprises B and/or T cell DNA and said one or more target nucleotide sequences is selected from:
(i) one or more rearranged V, D or J gene segments;
(ii) the DJ or VDJ rearrangements of IgH, TCR β or TCR δ;
(iii) a kappa deleting element rearrangement;
(iv) the VJ rearrangement of Igκ, Igλ, TCRα or TCRγ;
(v) a V gene segment region such s a region predisposed to undergoing hypermutation and/or a J gene segment region encoding a portion of the CDR3:
(vi) the gene segment regions encoding all or some of the V leader sequence, the V region predisposed to somatic hypermutation, IgH FR1, IgH FR2 or IgH FR3; and/or
(vii) the BCL1/JH or BCL2/JH translocation or an internal tandem duplication or other mutation associated with the FLT3 or TP53 genes.

5.-9. (canceled)

10. The method according to claim 1 wherein said solid support is a glass surface, such as glass slide or a flow cell.

11. (canceled)

12. The method according to claim 1 wherein said template DNA molecule expresses one or more nucleic acid sequences corresponding to indexes, barcodes, unique molecular identifiers, sequencing primer hybridisation sites and index sequencing primer hybridisation sites at the terminal 5′ and/or 3′ position.

13. The method according to claim 1 wherein said contiguous nucleotide region of step (i) corresponds to about 80% of the maximum forward and reverse read length deliverable by the bidirectional sequencing technology selected for use in step (iii)

14. The method according to claim 1 wherein said contiguous nucleotide region corresponds to 75%, 76%, 77%, 78%, 79%, 80%, 81%, 82% or 83% of the maximum forward and reverse read length deliverable by the bidirectional sequencing technology selected for use in step (iii) and said forward and reverse read portions is not less than 75%, 76%, 77%, 78%, 79%, 80%, 81%, 82% or 83% of the maximum forward and reverse read length deliverable by the bidirectional sequencing technology selected for use in step (iii).

15. The method according to claim 14 wherein said target DNA sequences are:
(i) localised to the 120 contiguous nucleotides at the 5′ and/or 3′ terminal ends of said template but wherein the 20 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites; or
(ii) localised to the 125 contiguous nucleotides at the 5′ and/or 3′ terminal ends of said template but wherein up to the 30 nucleotide terminal ends of said contiguous nucleotide region express one or more nucleotide sequences corresponding to adaptors, indexes, barcodes, unique molecular identifiers, sequencing primer hybridisation sites or index sequencing primer hybridisation sites.

16. (canceled)

17. The method according to claim 1 wherein said amplification is bridge amplification and/or said method is sequencing by synthesis using reversibly terminated labelled nucleotides.

18. (canceled)

19. The method according to claim 1 wherein said nucleic acid linker is 5-30 nucleotides in length, preferably 5-25, more preferably 5-20 and still more preferably 5, 6, 7, 8, 9, 10, 11, 12, 13, 14, 15 or 16 nucleotides in length.

20. (canceled)

21. The method according to claim 1 wherein said analysis comprises aligning the nucleic acid sequence results generated in step (iv) and determining the expression of the target nucleic acid sequences of interest.

22. The method according to claim 2 wherein said condition is characterised by a clonal population of cells or microorganisms, preferably clonal lymphoid cells.

23. (canceled)

24. The method according to claim 2 wherein said condition is characterised by one or more target nucleotide sequences which are expressed by an immune cell, preferably one or more rearranged V, D or J gene segment sequence characteristics.

25. (canceled)

26. The method according to claim 24 wherein said condition is infection, transplantation, autoimmunity, immunodeficiency, allergy, neoplasia, such as a lymphoid or myeloid neoplasia, or any other condition characterised by T or B cell clonal expansion.

27. (canceled)

28. The method according to claim 26 wherein said condition is acute lymphoblastic leukaemia, acute lymphocytic leukaemia, acute myeloid leukemia, acute promyelocytic leukemia, chronic lymphocytic leukaemia, chronic myeloid leukemia, myeloproliferative neoplasms, such as myeloma, systemic mastocytosis, lymphoma or hairy cell leukemia, transplant rejection, immunotherapy, polycythemia vera, myelodysplasia and leucocytosis, such as lymphocytic leucocytosis.

29. The method according to claim 26 wherein said method is used to detect minimum residual disease.

30.-31. (canceled)

32. The method according to claim 2 wherein said method is applied to diagnosis, prognosis, prediction of disease risk, detection of recurrence of disease, immune surveillance or monitoring prophylactic or therapeutic efficacy.

33. A computer-implemented method for preparing nucleic acid sequence results for analysis from non-overlapping sequence reads comprising:
identifying forward sequence reads and reverse sequence reads from sequence reads of a cluster of amplicons wherein the cluster is generated from an individual spatially isolated template DNA molecule, and each sequence read is generated by a selected bidirectional sequencing technology, and wherein the forward sequence reads and the reverse sequence reads do not overlap and do not provide a contiguous read across the full length of any amplicon; and
linking the forward sequence reads with the reverse sequence reads resulting in a plurality of first nucleic acid sequence results, such that each forward sequence read is linked to a reverse sequence read and each reverse sequence read is linked to a forward sequence read through a first nucleic acid linker sequence, wherein each linking is achieved by:
concatenating the first nucleic acid linker sequence between the 3′ end of a portion of the terminal 5′ contiguous nucleic acid sequence of a forward sequence read and the reverse complement of a portion of the terminal 5′ contiguous nucleic acid sequence of a reverse sequence read, thereby producing a first nucleic acid sequence result comprising the portion of the forward sequence read, the first nucleic acid linker sequence, and the reverse complement of the portion of the reverse sequence read in that order;
wherein (1) the length of the portion from the forward sequence read is not less than 75% of the maximum read length deliverable by the selected bidirectional sequencing technology, the length of the portion from the reverse sequence read is not less than 75% of the maximum read length deliverable by the selected bidirectional sequencing technology; (2) the length of the portion from the reverse sequence read is the same for all reverse sequence reads which are analysed; (3) the length of the portion from the forward sequence read is the same for all forward sequence reads which are analysed but may be the same or different to the length of the portion from the reverse sequence read and (4) the first nucleic acid linker sequence is the same for all first nucleic acid sequence results.

34. The computer-implemented method of claim 33, further comprising:
linking the forward sequence reads with the reverse sequence reads resulting in a plurality of second nucleic acid sequence results, such that each forward sequence read is linked to a reverse sequence read and each reverse sequence read is linked to a forward sequence read through a second nucleic acid linker sequence, wherein each linking is achieved by
concatenating the second nucleic acid linker sequence between the 3′ end of a portion of the terminal 5′ contiguous nucleic acid sequence of a reverse sequence read and the reverse complement of a portion of the terminal 5′ contiguous nucleic acid sequence of a forward sequence read, thereby producing a second nucleic acid sequence result comprising the portion from the reverse sequence read, the second nucleic acid linker sequence and the reverse complement of the portion from the forward sequence read in that order;
wherein (1) the length of the portion from the forward sequence read is not less than 75% of the maximum read length deliverable by the selected bidirectional sequencing technology, the length of the portion from the reverse sequence read is not less than 75% of the maximum read length deliverable by the selected bidirectional sequencing technology; (2) the length of the portion from the reverse sequence read being concatenated to the second nucleic acid linker is the same for all reverse sequence reads and is the same as the length of the portion from the reverse sequence read being concatenated to the first nucleic acid linker; (3) the length of the portion from the forward sequence read being concatenated to the second nucleic acid linker is the same for all forward sequence reads and is the same as the length of the portion from the forward sequence read being concatenated to the first nucleic acid linker, but may be the same or different to the length of the portion from the reverse sequence read being concatenated to the second nucleic acid linker, and (4) the second nucleic acid linker sequence is the same for all second nucleic acid sequence results.

35. The computer-implemented method of claim 34, wherein the first nucleic acid linker sequence and the second nucleic acid linker sequence are at least 11 nucleotides long.

36. The computer-implemented method of claim 33, wherein the length of the portion of the forward sequence read is the same as the length of the portion of the reverse sequence read.

37. The computer-implemented method of claim 33, wherein the portion of the forward sequence read comprises a specified number of contiguous nucleotides of the 5′ terminus of the forward sequence read, and the portion of the reverse sequence read comprises a specified number of contiguous nucleotides of the 5′ terminus of the reverse sequence read, preferably wherein the specified number of contiguous nucleotides comprises between about 80 nucleotides and about 180 nucleotides.

38.-39. (canceled)

40. The computer-implemented method of claim 33, wherein the cluster of amplicons is amplified from B and/or T cell DNA and preferably comprises at least one rearranged V, D or J gene segment.

41. (canceled)

42. A non-transitory computer-readable storage medium having program instructions embodied therewith, the program instructions executable by a processing element of a device to cause the device to implement a method for preparing nucleic acid sequence results for analysis from non-overlapping sequence reads by:
identifying forward sequence reads and reverse sequence reads from sequence reads of a cluster of amplicons wherein the cluster is generated from an individual spatially isolated template DNA molecule, and each sequence read is generated by a selected bidirectional sequencing technology, and wherein the forward sequence reads and the reverse sequence reads do not overlap and do not provide a contiguous read across the full length of any amplicon; and
linking the forward sequence reads with the reverse sequence reads resulting in a plurality of first nucleic acid sequence results, such that each forward sequence read is linked to a reverse sequence read and each reverse sequence read is linked to a forward sequence read through a first nucleic acid linker sequence, wherein each linking is achieved by:
concatenating the first nucleic acid linker sequence between the 3′ end of a portion of the terminal 5′ contiguous nucleic acid sequence of a forward sequence read and the reverse complement of a portion of the terminal 5′ contiguous nucleic acid sequence of a reverse sequence read, thereby producing a first nucleic acid sequence result comprising the portion of the forward sequence read, the first nucleic acid linker sequence, and the reverse complement of the portion of the reverse sequence read in that order;
wherein (1) the length of the portion from the forward sequence read is not less than 75% of the maximum read length deliverable by the selected bidirectional sequencing technology, the length of the portion from the reverse sequence read is not less than 75% of the maximum read length deliverable by the selected bidirectional sequencing technology; (2) the length of the portion from the reverse sequence read is the same for all reverse sequence reads which are analysed; (3) the length of the portion from the forward sequence read is the same for all forward sequence reads which are analysed but may be the same or different to the length of the portion from the reverse sequence read and (4) the first nucleic acid linker sequence is the same for all first nucleic acid sequence results.

43. The non-transitory computer-readable storage medium of claim 42, further comprising:
linking the forward sequence reads with the reverse sequence reads resulting in a plurality of second nucleic acid sequence results, such that each forward sequence read is linked to a reverse sequence read and each reverse sequence read is linked to a forward sequence read through a second nucleic acid linker sequence, wherein each linking is achieved by
concatenating the second nucleic acid linker sequence between the 3′ end of a portion of the terminal 5′ contiguous nucleic acid sequence of a reverse sequence read and the reverse complement of a portion of the terminal 5′ contiguous nucleic acid sequence of a forward sequence read, thereby producing a second nucleic acid sequence result comprising the portion from the reverse sequence read, the second nucleic acid linker sequence and the reverse complement of the portion from the forward sequence read in that order;
wherein (1) the length of the portion from the forward sequence read is not less than 75% of the maximum read length deliverable by the selected bidirectional sequencing technology, the length of the portion from the reverse sequence read is not less than 75% of the maximum read length deliverable by the selected bidirectional sequencing technology; (2) the length of the portion from the reverse sequence read being concatenated to the second nucleic acid linker is the same for all reverse sequence reads and is the same as the length of the portion from the reverse sequence read being concatenated to the first nucleic acid linker; (3) the length of the portion from the forward sequence read being concatenated to the second nucleic acid linker is the same for all forward sequence reads and is the same as the length of the portion from the forward sequence read being concatenated to the first nucleic acid linker, but may be the same or different to the length of the portion from the reverse sequence read being concatenated to the second nucleic acid linker, and (4) the second nucleic acid linker sequence is the same for all second nucleic acid sequence results.

44. The non-transitory computer-readable storage medium of claim 42, wherein the first nucleic acid linker sequence and the second nucleic acid linker sequence are at least 11 nucleotides long.

45. The non-transitory computer-readable storage medium of claim 42, wherein the length of the portion of the forward sequence read is the same as the length of the portion of the reverse sequence read.

46. The non-transitory computer-readable storage medium of claim 42, wherein the portion of the forward sequence read comprises a specified number of contiguous nucleotides of the 5′ terminus of the forward sequence read, and the portion of the reverse sequence read comprises the specified number of contiguous nucleotides of the 5′ terminus of the reverse sequence read, preferably wherein the specified number of contiguous nucleotides comprises between about 80 nucleotides and about 180 nucleotides.

47.-48. (canceled)

49. The non-transitory computer-readable storage medium of claim 42, wherein the cluster of amplicons is amplified from B and/or T cell DNA and preferably comprises at least one rearranged V, D or J gene segment.

50. (canceled)

51. A device for preparing nucleic acid sequence results for analysis from non-overlapping sequence reads, comprising:
a hardware processor being configured to:
identify forward sequence reads and reverse sequence reads from sequence reads of a cluster of amplicons wherein the cluster is generated from an individual spatially isolated template DNA molecule, and each sequence read is generated by a selected bidirectional sequencing technology, and wherein the forward sequence reads and the reverse sequence reads do not overlap and do not provide a contiguous read across the full length of any amplicon; and
link the forward sequence reads with the reverse sequence reads resulting in a plurality of first nucleic acid sequence results, such that each forward sequence read is linked to a reverse sequence read and each reverse sequence read is linked to a forward sequence read through a first nucleic acid linker sequence, wherein each linking is achieved by:
concatenating the first nucleic acid linker sequence between the 3′ end of a portion of the terminal 5′ contiguous nucleic acid sequence of a forward sequence read and the reverse complement of a portion of the terminal 5′ contiguous nucleic acid sequence of a reverse sequence read, thereby producing a first nucleic acid sequence result comprising the portion of the forward sequence read, the first nucleic acid linker sequence, and the reverse complement of the portion of the reverse sequence read in that order;
wherein (1) the length of the portion from the forward sequence read is not less than 75% of the maximum read length deliverable by the selected bidirectional sequencing technology, the length of the portion from the reverse sequence read is not less than 75% of the maximum read length deliverable by the selected bidirectional sequencing technology; (2) the length of the portion from the reverse sequence read is the same for all reverse sequence reads which are analysed; (3) the length of the portion from the forward sequence read is the same for all forward sequence reads which are analysed but may be the same or different to the length of the portion from the reverse sequence read and (4) the first nucleic acid linker sequence is the same for all first nucleic acid sequence results.

52. The device of claim 51, wherein the hardware processor is further configured to:
link the forward sequence reads with the reverse sequence reads resulting in a plurality of second nucleic acid sequence results, such that each forward sequence read is linked to a reverse sequence read and each reverse sequence read is linked to a forward sequence read through a second nucleic acid linker sequence, wherein each linking is achieved by
concatenating the second nucleic acid linker sequence between the 3′ end of a portion of the terminal 5′ contiguous nucleic acid sequence of a reverse sequence read and the reverse complement of a portion of the terminal 5′ contiguous nucleic acid sequence of a forward sequence read, thereby producing a second nucleic acid sequence result comprising the portion from the reverse sequence read, the second nucleic acid linker sequence and the reverse complement of the portion from the forward sequence read in that order;
wherein (1) the length of the portion from the forward sequence read is not less than 75% of the maximum read length deliverable by the selected bidirectional sequencing technology, the length of the portion from the reverse sequence read is not less than 75% of the maximum read length deliverable by the selected bidirectional sequencing technology; (2) the length of the portion from the reverse sequence read being concatenated to the second nucleic acid linker is the same for all reverse sequence reads and is the same as the length of the portion from the reverse sequence read being concatenated to the first nucleic acid linker; (3) the length of the portion from the forward sequence read being concatenated to the second nucleic acid linker is the same for all forward sequence reads and is the same as the length of the portion from the forward sequence read being concatenated to the first nucleic acid linker, but may be the same or different to the length of the portion from the reverse sequence read being concatenated to the second nucleic acid linker, and (4) the second nucleic acid linker sequence is the same for all second nucleic acid sequence results.

53. The device of claim 52, wherein the first nucleic acid linker sequence and the second nucleic acid linker sequence are at least 11 nucleotides long.

54. The device of claim 51, wherein the length of the portion of the forward sequence read is the same as the length of the portion of the reverse sequence read.

55. The device of claim 51, wherein the portion of the forward sequence read comprises a specified number of contiguous nucleotides of the 5′ terminus of the forward sequence read, and the portion of the reverse sequence read comprises the specified number of contiguous nucleotides of the 5′ terminus of the reverse sequence read, preferably wherein the specified number of contiguous nucleotides comprises between about 80 nucleotides and about 180 nucleotides.

56.-57. (canceled)

58. The device of claim 51, wherein the cluster of amplicons is amplified from B and/or T cell DNA and preferably comprises at least one rearranged V, D or J gene segment.

59. (canceled)


SEQUENCE LISTING (10 sequences)

SEQ ID NO: 1 - DNA, 37 bp, Artificial Sequence
  Oligonucleotide
  misc_feature (16)..(26): n is a, c, g, or t
gctatgcggactctgnnnnnnnnnnngccaagaactc

SEQ ID NO: 2 - DNA, 36 bp, Artificial Sequence
  Oligonucleotide
  misc_feature (15)..(25): n is a, c, g, or t
gctatgcggactctnnnnnnnnnnngccaagaactc

SEQ ID NO: 3 - DNA, 33 bp, Artificial Sequence
  Oligonucleotide
  misc_feature (12)..(22): n is a, c, g, or t
gctatgcggacnnnnnnnnnnngccaagaactc

SEQ ID NO: 4 - DNA, 37 bp, Artificial Sequence; identical to SEQ ID NO: 1
  Oligonucleotide
  misc_feature (16)..(26): n is a, c, g, or t

SEQ ID NO: 5 - DNA, 36 bp, Artificial Sequence; identical to SEQ ID NO: 2
  Oligonucleotide
  misc_feature (15)..(25): n is a, c, g, or t

SEQ ID NO: 6 - DNA, 32 bp, Artificial Sequence
  Oligonucleotide
  misc_feature (11)..(23): n is a, c, g, or t
gattgggatgnnnnnnnnnnnnnccaggtggt

SEQ ID NO: 7 - DNA, 30 bp, Artificial Sequence
  Oligonucleotide
  misc_feature (11)..(23): n is a, c, g, or t
gattgggatgnnnnnnnnnnnnnaggtggt

SEQ ID NO: 8 - DNA, 33 bp, Artificial Sequence
  Oligonucleotide
  misc_feature (11)..(23): n is a, c, g, or t
gattgggatgnnnnnnnnnnnnnaccaggtggt

SEQ ID NO: 9 - DNA, 31 bp, Artificial Sequence
  Oligonucleotide
  misc_feature (11)..(23): n is a, c, g, or t
gattgggatgnnnnnnnnnnnnncaggtggt

SEQ ID NO: 10 - DNA, 30 bp, Artificial Sequence; identical to SEQ ID NO: 7
  Oligonucleotide
  misc_feature (11)..(23): n is a, c, g, or t